(19) United States
(12) Patent Application Publication
(10) Pub. No.: US 2026/0308073 A1
(43) Pub. Date: Oct. 8, 2026

(54) METHODS TO CHARACTERIZE, TREAT AND PROCESS BIOCARBON(S) AND COMBINATIONS THEREOF AS A CONCRETE INGREDIENT REPLACER AND CONCRETE PERFORMANCE ENHANCER AND PREDICTOR

(71) Applicant: ECOLOCKED GMBH, Berlin (DE)

(72) Inventors: Micheil GORDON, Berlin (DE); Gilberto NERY DE ARAUJO NETO, Berlin (DE); Nsesheye Susan PRIEBE, Berlin (DE); Rawia DABBEBI, Berlin (DE); Feras TAJRA, Berlin (DE)

(21) Appl. No.: 19/482,588

(22) PCT Filed: May 10, 2024
(86) PCT No.: PCT/EP2024/062948

§ 371 Date: Nov. 7, 2025

Related U.S. Application Data
(60) Provisional application No. 63/465,086, filed on May 9, 2023.

(51) Int. Cl. C04B 40/00; C04B 14/02; C04B 24/34; C04B 28/08; C04B 103/32; C04B 111/00

(52) U.S. Cl. CPC C04B 40/0032; C04B 14/022; C04B 24/34; C04B 28/08; C04B 40/0046; C04B 2103/32; C04B 2111/00068; C04B 2201/50

(57) ABSTRACT

A method of producing a concrete mixture with at least one biocarbon. The method may include determining a set of parameters for processing the at least one biocarbon and/or the concrete mixture based on a biocarbon water correction factor (BWCF) of the at least one biocarbon, which is defined as a percentage of a water absorption of the at least one biocarbon. The biocarbon water correction factor is preferably in a range of to 100% of the water absorption of the at least one biocarbon, more preferred 40 to 70% of the water absorption when the concrete mixture is a flowable concrete mixture or 20 to 50% of the water absorption, when the concrete mixture is an earth moist concrete mixture.

### BACKGROUND OF THE DISCLOSURE

### 1. Field of the Disclosure

The present disclosure relates to cementitious materials, concrete compositions and characteristics, and methods of making and improving them. More particularly, the present disclosure relates to a method of producing a concrete mixture, a concrete mixture, a customized biocarbon, the use of a biocarbon in a concrete mixture, an optimization tool and a computer readable medium.

### 2. Discussion of the Related Art

Concrete is the second most consumed material on Earth, with global production totaling more than four billion metric tons per year. The production of cement generates significant CO₂ emissions, equivalent to approximately 8% of the total global greenhouse gas emissions. The CO₂ release occurs from two main sources, namely from the decomposition of carbonates found in limestone (used as a cementitious material), and from the fossil-derived energy required for this decomposition. Without concrete and cement decarbonization solutions, the associated emissions are expected to double by 2060. While the construction sector has committed to reducing embodied emissions to net zero by 2050, there is a lack of cost-effective and scalable approaches to reach that goal.

Scalable and economical carbon dioxide removal (CDR) technologies are limited, however, and their large-scale deployment will take too long to reduce the atmospheric CO₂ concentration in time to contain global warming at 1.5-2 degrees. Biocarbon (also known as biochar) production provides one of the most economical CDR technologies available and scalable today. Furthermore, biocarbon can provide some of the highest permanence carbon sequestration solutions if provided with a stable storage solution such as within concrete. Recently, biocarbon has been gaining increasing attention among the scientific community as a decarbonization solution for construction materials. Biocarbon, also known as biochar, is often described as a biomass-derived carbon-rich and carbon-stable material produced through the thermal decomposition (e.g. pyrolysis) of biomass feedstocks at moderate temperatures. The resulting material, biocarbon, is far more stable than the biomass input feedstocks and can prevent the release of emissions for hundreds to thousands of years.

Biocarbon materials' physicochemical, structural, and microstructural properties vary, and are dependent on the biocarbon source feedstock, processing methods and technology, and specific processing parameters such as heating rate, residence time, temperature range, and oxygen availability. The varying parameters also affect the final mechanical properties of the biocarbon such as compressive and impact strengths as well as the Young's Modulus and hardness which in turn affects its performance within the resulting use materials, concrete. The variability across biocarbon materials poses a challenge for concrete integration, where material uniformity and performance predictability are high priorities. There are no currently available resources to assist in this goal. The present disclosure addresses these needs.

There are many technological challenges in biocarbon selection and integration for cementitious systems, which include:
a. Biocarbon inhomogeneity and material suitability
Biocarbon materials' physicochemical, structural, and microstructural properties vary, and are dependent on the biocarbon source feedstock, processing methods and technology, and specific processing parameters such as heating rate, residence time, temperature range, and oxygen availability. The varying parameters also affect the final mechanical properties of the biocarbon such as compressive and impact strengths as well as the Young's Modulus and hardness. The variability across biocarbon materials poses a challenge for concrete integration, where material uniformity and performance predictability are high priorities.
b. Lack of predictive tools for multi-biocarbon type use in cementitious systems
There are no tools to predict biocarbon's performance in cementitious systems for multiple biocarbon utilization options and towards biocarbon performance classifications and clustering.
c. Biocarbon material stability in the cement matrix
The current lack of understanding of the drivers for long-term concrete performance and stability is a barrier to the use of biocarbon materials for concrete applications.
d. Compromised fresh concrete performance
The rheology of biocarbon amended concrete mix designs is highly variable. Different biocarbon materials can lead to a significant loss to fresh concrete workability, increased segregation and/or bleeding.
e. Reduction in hardened concrete mechanical performance
Biocarbons may cause a reduction in the flexural and compressive strength of the concrete. They also have an unclear effect on the concrete (due to chloride ingress, depth of water penetration/permeability, freeze thaw, acid attack, carbonation, water absorption through capillarity).
f. Biocarbons may not always be compatible with other concrete decarbonization technologies, i.e., biocarbon technology stacking such as the combination of biocarbon with the following developed and emerging technologies:
   Low CO₂ SCMs (fly ash, blast furnace slag, calcined clays etc.)
   CO₂ concrete curing
   CO₂ mineralization/biomineralization
   Recycled aggregates
   Biogenic limestone
g. There is limited knowhow regarding the drivers of concrete functional benefits, such as:
   Certified CO₂ footprint reduction
   Reduced thermal and acoustic conductivity
   Improved mechanical strength performance (flexural, compressive strength)
   Improved temperature/fire resistance
   Weight reduction
   Improved durability
h. It is unclear how to measure CO₂ removal, along with reporting and verification.
i. There is no current guidance on the challenges of biocarbon-to-concrete integration, such as:
   Material preparation requirements, from lab to production scale
   Biocarbon material integration at the concrete production plant/onsite mixing/batching
   Material handling challenges for transportation and delivery, such as material powder flowability, dust explosion risks, and moisture content management and variability
j. The interaction(s) of biocarbon(s) and chemical additives, usually used for cementitious systems. Specifically, biocarbon(s) may alter (increase or decrease) chemical additive effectiveness to perform their desired function on the cementitious systems.

The present disclosure addresses some of these disadvantages.

### SUMMARY OF THE DISCLOSURE

According to a first aspect the invention relates to a method of producing a concrete mixture comprising at least one biocarbon, the method comprising determining a set of parameters for processing the at least one biocarbon and/or the concrete mixture based on a biocarbon water correction factor (BWCF) of the at least one biocarbon, which is defined as a percentage of a water absorption of the at least one biocarbon, wherein the biocarbon water correction factor is preferably in a range of 10 to 100% of the water absorption of the at least one biocarbon, more preferred 40 to 70% of the water absorption, when the concrete mixture is a flowable concrete mixture or 20 to 50% of the water absorption, when the concrete mixture is an earth moist concrete mixture. The biocarbon water correction factor BWCF is in other words the product of a percentage, namely the biocarbon water correction percentage BWCP, and the water absorption WA of the at least one biocarbon, thus BWCF=BWCP * WA. The biocarbon water correction factor multiplied with the dry weight of the at least one biocarbon in the mixture gives the weight of an amount of water to add into the concrete mixture to compensate the presence of the biocarbon. Thus the biocarbon water correction factor indicates an amount of water to add into the concrete mixture to compensate the presence of the biocarbon as percentage of the weight of the biocarbon.

This method is preferably a computer-implemented method. By using the biocarbon water correction factor the concrete mixture can be optimized.

The invention is based on the recognition that in order to provide concrete mixtures, which on one hand contribute to a CO₂ reduction in the atmosphere as desired by using biocarbon in the concrete mixture and on the other hand also show high performance, it is especially useful to compensate the presence of the biocarbon by adding more water into the concrete mixture in comparison to the case of a concrete mixture without biocarbon. The invention thus includes the recognition that compensating the presence of biocarbon by adding more water to the concrete mixtures avoids a debased performance of the concrete mixture regarding for example mechanical strength or flowability due to the presence of biocarbon.

The invention further includes the recognition that this amount of water is especially dependent on the water absorption of the used biocarbon. Preferable performance of concrete mixture can be reached especially with the biocarbon water correction factor being in the range of 10 to 100% of the water absorption of the biocarbon. The invention is further based on the recognition that there are fixed percentages of the water absorption, which are favorable as biocarbon water correction percentage for biocarbons.

The invention further includes the recognition that the water absorption of the biocarbon and thus also the biocarbon water correction factor are dependent on further properties of the biocarbon, such as for example the particle size distribution. Thus the needed amount of water or the biocarbon water correction factor can be influenced and changed during processing of the biocarbon. The invention is based on the recognition that for a concrete mixture several properties of the biocarbon and process parameter of the mixture have to be taken into account and that with the use of the biocarbon water correction factor their complex interplay on the performance of the concrete mixture can be described and be controlled in a manageable way. Thus as the inventors have recognized the consideration of the biocarbon water correction factor when composing a concrete mixtures allows to optimize the overall performance of concrete mixture while integrating biocarbon.

The water absorption is given in mass percent and gives the weight of water absorbed by the respective water saturated and surface dry biocarbon per selfweight of the surface-dry biocarbon and can be determined according to EN 13055.

The invention further includes the recognition that taking into account the biocarbon water correction factor surprisingly allows for higher strength of the final concrete mixture despite dosages of biocarbon in relatively high ranges. Thus the invention as a whole allows for performance-competitive concrete mixtures with relatively high strength and considerable CO₂ equivalent storage potential.

The invention is further based on the recognition that the biocarbon water correction factor as being dependent on the water absorption is also dependent on further material properties of the biocarbon, such as for example the median particle size. Thus by processing the biocarbon for example to a changed median particle size the value of the water absorption and thus the biocarbon water correction factor will also change. On the other hand the median particle size of a biocarbon itself also does influence performance and workability of a concrete mixture with that biocarbon. Thus the production of a concrete mixture comprising at least one biocarbon is a complex procedure, in which several parameter and properties and their influence have to be weighted. The invention has recognized that this complex interaction can be approximated and the concrete mixture improved in basing the determination of the process parameters for processing the at least one biocarbon and the concrete mixture on the biocarbon water correction factor.

The invention has recognized that the addition of water according to the biocarbon water correction factor is one key to improve the performance of concrete mixtures with biocarbon. Preferably further relationships between performance of the concrete mixture and material properties of the biocarbon or other production parameters are taken into account as outlined below.

The present disclosure offers a comprehensive solution to the challenges of reducing the carbon footprint of concrete production while maintaining optimal mechanical performance. The methods disclosed herein involve the selection of one or more biocarbon(s) as a concrete ingredient replacer, according to the chemical and morphological properties of the biocarbon, which allows for the prediction and control of the performance of the new concrete. Additionally, the method includes biocarbon-amended concrete mixture optimizations for long-term CO₂ equivalent storage and overall CO₂ footprint reduction. Biocarbon blends including the use of additives as well as pre-treatment methods for further performance enhancements are also included.

The present disclosure provides extremely helpful tools for integrating biocarbons into concrete mixtures and at the same time accounting for performance. There are no currently available systems or analytical tools that can determine the needs of a particular mix or application and determine suitable biocarbon replacements for the inorganic components in the mix, exactly what properties the replacement biocarbon should have, and develop a revised and optimized concrete mixture.

It is another aspect of the present disclosure that a correlation exists between the biocarbon material characteristics and the level of the water content, sometimes referred to as moisture or the moisture content, which influences the water absorption that can be held within the biocarbon material during the mixing process. Consequently, the properties to influence the final concrete and/or mortar properties of the different biocarbon materials can be correlated to their interaction with water, preferably with their water holding capacity and/or water absorption.

The consideration of the biocarbon water correction factor provides the additional benefits for the concrete mixture: increased workability without compromising 7/28-day strength performance, reducing the required dose of superplasticizer, increased mix moisture to provide more efficient binder use for later stage hydration reactions and for increased later stage strength developments, i.e. provides internal curing.

The biocarbon water correction factor is calculated from the water absorption of the used biocarbon material and thus reflects and is also based on one or a combination thereof the following biocarbon material characteristics: water holding capacity (WHC), specific surface area (SSA), particle size distribution and median particle size d50, feedstock, pyrolysis temperature.

As used in the present disclosure, the term “concrete” refers to any cementitious based system comprising a combination of cementitious based binders with aggregates, and optionally water and optionally additives. Cementitious based binders include but are not limited to silicate cements, Portland cements, CEM I, CEM II, CEM III, CEM IV, CEM V, according to EN 197-1, and CEM VI. Aggregates include but are not limited to, gravel, silt, sand, fines, light weight aggregates; and further according to EN 12620 and/or EN 13055.

As used in the present disclosure, “biocarbon” refers to the solid product from the thermo-chemical decomposition of biomass at moderate temperatures under oxygen-controlled conditions. The resulting solid product known as biocarbon or sometimes as biochar, is far more stable than the biomass input feedstocks and can prevent the release of emissions for hundreds to thousands of years. In one aspect, the biocarbon of the present disclosure contains a minimum of 20 wt. % of carbon based on the elemental composition of the entire biocarbon material measured according to DIN 51732 or ISO 16948:2015. In other aspects, the biocarbon contains a minimum of 30 wt. %, minimum of 40 wt. % measured according to DIN 51732 or ISO 16948:2015, minimum of 50 wt. % measured according to DIN 51732 or ISO 16948:2015, minimum of 60 wt. % measured according to DIN 51732 or ISO 16948:2015, minimum of 70 wt. % measured according to DIN 51732 or ISO 16948:2015, a minimum of 75 wt. % measured according to DIN 51732 or ISO 16948:2015, a minimum of 80 wt. % measured according to DIN 51732 or ISO 16948:2015, minimum of 85 wt. % measured according to DIN 51732 or ISO 16948:2015, a minimum of 90 wt. % measured according to DIN 51732 or ISO 16948:2015, or a minimum of 95 wt. % carbon based on the elemental composition of the entire biocarbon material measured according to DIN 51732 or ISO 16948:2015.

As used in the present disclosure, “flowable concrete” refers to all wet mix concrete that falls into a slump class or flow class according to the definition outlined in EN 206. As used in the present disclosure, “workability” refers to the rheological performance measured by the class of slump and/or flow of wet mix concrete that falls into a slump class or flow class according to the definitions outlined in EN 206.

As used in the present disclosure, “earth moist concrete” refers to a type of concrete mixture characterized by its low moisture content, which is just sufficient to hydrate the cement and hold the mixture together without any excess water. This consistency allows the concrete to be compacted into molds or forms with mechanical pressure instead of relying on the fluidity of wetter concretes.

The thermal decomposition processes can include, but are not limited to the following presented in Table 1 and any combinations thereof:

TABLE 1
Thermal decomposition processes and parameters

| Thermal | | | |
|---|---|---|---|
| decomposition | Temperature | | |
| processes | range | Heating rate | Other |
| slow pyrolysis | 300 °C to 600 °C | 5 °C to 7 °C per min | |
| fast pyrolysis | >500 °C | 300 °C per min | |
| flash pyrolysis | 900 °C to 1200 °C | 800 °C to 1000 °C | |
| | | per min | |
| vacuum pyrolysis | 450 °C to 600 °C | 300 °C per min | typical pressure of 0.05 |
| | | | MPa to 0.20 Mpa |
| microwave | 450 °C to 800 °C | | typical microwave power |
| pyrolysis | | | of 400 W to 600 W and a |
| | | | frequency of 2450 MHZ |
| torrefaction | 230 °C to 300 °C | | |
| gasification | >700 °C | | and with a gasifying agent, |
| | | | typically steam |
| hydrothermal | 220 °C to 240 °C | | typical pressure of 2 Mpa |
| carbonization | | | to 10 Mpa |

In Table 2a certain physical properties of biocarbon and the related norm or method for their determination are listed.

TABLE 2a
Physical properties of biocarbon and their relevant standards

| Property | Related Standard |
|---|---|
| Carbon content [%] | DIN 51732 |
| Ash content | DIN 51719 |
| Random reflectance | ISO 7404-5 |
| Total water content [%]: | EN 1097-5 |
| Water absorption [%]: | EN 13055 |
| Water holding capacity [%] | ISO 14238 |
| Particle size | ISO 19749 |
| Particle shape | ISO 19749 |
| Particle size distribution | EN 933-1 |
| Median particle size | |
| Specific surface area [m²/g] | ISO 9277 |
| Loose bulk density [kg/m³]: | EN 1097-3 |
| Particle density [kg/m³]: | EN 13055 |
| Water-soluble chloride ion [%]: | EN 1744-1 |
| Acid-soluble sulphate [%] | EN 1744-1 |
| Total sulphur [%] | EN 1744-1 |
| Fire resistance | EN 13501-1 |

In Table 2b the relevant standards for properties of concrete mixtures mentioned in the present disclosure are listed

TABLE 2b
Relevant standards

| Property | Relevant standard |
|---|---|
| Flow table test | EN 12350-5 |
| Fresh density | EN 12350-6 |
| Air content test | EN 12350-7 |
| Compressive Strength | EN 12390-3 |
| Dry Density | EN 12390-7 |
| Carbonation | EN 13295 |
| Freeze thaw | CEN/TS 12390-9 |
| Water permeability (capillary water absorption) | EN ISO 15148 |
| Alkali-Silica reaction | ASTM C1260 |
| Shrinkage | EN 12390-16 |
| Fire/heat resistance | EN 13823 |
| Chloride permeability | ASTM C1202 |
| Acid attack | ASTM C1898-20 |

Preferably the method further comprises the step of providing the set of parameters as preferably machine-readable data for controlling the processing of the at least one biocarbon and/or the concrete mixture. Preferably the method comprises the step of controlling the processing of the at least one biocarbon and/or the concrete mixture with the set of parameters or based on the set of parameters.

In an embodiment the biocarbon water correction percentage for a flowable concrete is in the range of 40 to 65%, more preferred 45 to 65%, more preferred 50 to 60%, more preferred 55 to 60%, more preferred 57 to 58%.

In a further embodiment the biocarbon water correction percentage for an earth moist concrete is in the range of 20 to 45%, more preferred 25 to 45%, more preferred 30 to 40%, more preferred 35 to 40%.

In a preferred embodiment the method comprises before determining the set of parameters the step of determining the water absorption of the at least one biocarbon.

In a preferred embodiment the water absorption is determined by measuring or calculating or estimating taking into account a water holding capacity, a water content, a median particle size and/or particle density of the at least one biocarbon. In order to determine the water absorption the water absorption can either be measured directly at the at least one biocarbon of the group of possible biocarbons. Alternatively the water absorption can also be calculated or estimated from other already known material properties of the at least one biocarbon, namely by using a water holding capacity, a water content, a median particle size and/or particle density. Determining the water absorption can also comprise deriving the water absorption from data provided by a manufacturer of the at least one biocarbon.

In a further embodiment the set of parameters comprises at least one selected from the group consisting of:
selection of at least one biocarbon from a group of possible biocarbons for the concrete mixture;
required median particle size,
I required water content of the at least one biocarbon before adding to the concrete mixture
process parameters for an adjustment of the median particle size,
process parameters for a pre-wetting of the at least one biocarbon,
dose of the selected at least one biocarbon,
required total amount of added water,
required amount of added water during mixing the concrete mixture,
required amount and type of added cement,
required amount and type of at least one additive,
required amount and type of at least one plasticizer, preferably of at least one superplasticizer,
aggregate replacement ratio,
a corrected water-cement ratio,
order of addition.

According to one or more of these parameters the at least one biocarbon and/or the concrete mixture can be subsequently further processed to get a concrete mixture. Starting from the biocarbon water correction factor especially the mentioned parameters can be determined. For example the at least one of at least one biocarbon from a group of possible biocarbons for the concrete mixture can be selected based on the water absorption of each biocarbon in the group, knowing that a higher water absorption leads to a higher biocarbon water correction factor and thus a higher amount of water to add per weight of the biocarbon in the concrete mixture, while the biocarbon water correction percentage stays constant.

As a first step of processing at least one biocarbon can be selected from a group of biocarbons, which are possible for the concrete mixture. Such possible biocarbons can be for example biocarbons available on site or with known further advantages for a specific use case of the concrete mixture.

As the median particle size also influences via the water absorption the biocarbon water correction factor, a required median particle size can be derived inter alia from the biocarbon water correction, e.g. BWCF can be lowered by adjusting the median particle. Thus also process parameters for an adjustment of the median particle size can be derived from the BWCF.

Also a required water content of the at least one biocarbon before adding to the concrete mixture can be derived from the BWCF, which gives the amount of water to compensate the presence of the biocarbon and at least a part of this water amount can be added to the at least one biocarbon before adding to the concrete mixture. This can be done via pre-wetting. Thus also process parameters for a pre-wetting of the at least one biocarbon can be derived from the BWCF.

The water content is an amount of water to be added to the amount of the dry weigth of the biocarbon before mixing. The water content is preferably determined as percentage of the water absorption of the at least one biocarbon, thus the water content is given as x % of the water absorption. In order to determine the mass of water to add the water content is multiplied with the mass of the at least one biocarbon.

As the BWCF gives the amount of water as percentage and can be used to calculate the needed water amount from the weight of the at least one biocarbon in the concrete mixture also a dose of the selected at least one biocarbon can be determined taking into account the BWCF. The same applies to the required total amount of added water, which is a sum of the water according to the BWCF and a water amount due to a targeted water-cement ratio for a reference concrete mixture without biocarbon. Such a reference concrete mixture is a concrete mixture without biocarbon, which has favorable performance parameters for an intended use case of the concrete mixture with biocarbon. In other words: a reference concrete mixture is herein understand as a concrete mixture without addition of biocarbon, which has targeted properties, that the concrete mixture optimized according to the method of the invention should also fulfill or is intended for despite using biocarbon.

Also a required amount of added water during mixing the concrete mixture can derived from the BWCF as this is the total amount of water minus the water content of the biocarbon before adding to the mixture.

The BWCF and the related amount of water also influence and thus determine further parameters and ingredients of the concrete mixtures such as required amount and type of added cement, required amount and type of at least one additive, required amount and type of at least one plasticizer, preferably of at least one superplasticizer, aggregate replacement ratio.

The corrected water-cement ratio gives the total water to cement ratio for the concrete mixture with at least one biocarbon. In other words the corrected water-cement ratio gives the ratio of total water amount-comprising the water needed for a targeted water-cement ratio of a reference concrete mixture without biocarbon and the water amount based on the biocarbon water correction factor—to cement amount in the concrete mixture. This parameter is thus also influenced by the BWCF and can especially be determined as follows.

The corrected water-cement ratio can be determined as

$${( \frac{w}{c} )}_{corr} = {( \frac{w}{c} )}_{t} \cdot ( 1 + \frac{B W C F \cdot {m}_{biocarbon}}{{w}_{t}} )$$

wherein BWCF is the biocarbon water correction factor,

$${( \frac{w}{c} )}_{t}$$

is a targeted water-cement ratio for a reference concrete mixture without biocarbon, m_{carbon} is the mass of biocarbon in the concrete mixture and wₜ is a targeted water amount for the reference concrete mixture without biocarbon. The corrected water-cement ratio can also be determined as

$${( \frac{w}{c} )}_{corr} = {( \frac{w}{c} )}_{t} \cdot ( 1 + \frac{B W C F \cdot {m}_{biocarbon}}{{{c}_{t} ( \frac{w}{c} )}_{t}} ) ,$$

wherein BWCF is the biocarbon water correction factor,

$${( \frac{w}{c} )}_{t}$$

is a targeted water-cement ratio for a reference concrete mixture without biocarbon, m_{biocarbon} is the mass of biocarbon in the concrete mixture and cₜ is a targeted cement amount for the reference concrete mixture without biocarbon.

The required total amount of water can be determined as wₜₒₜₐₗ=wₜ+BWCF·m_{biocarbon}, wherein BWCF is the biocarbon water correction factor, wₜ is a targeted water amount according to the targeted water-cement ratio for the reference concrete mixture without biocarbon

$${( \frac{w}{c} )}_{t}$$

and m_{biocarbon} is the mass of biocarbon in the concrete mixture.

The required amount of cement can be determined as

$$c = \frac{( {w}_{t} + B W C F \cdot {m}_{biocarbon} )}{{( \frac{w}{c} )}_{corr}} ,$$

wherein BWCF is the biocarbon water correction factor,

$${( \frac{w}{c} )}_{corr}$$

is the corrected water-cement ratio, m_{biocarbon} is the mass of biocarbon in the concrete mixture and wₜ is a targeted water amount for a reference concrete mixture without biocarbon.

With the formulas given above for the water-cement ratio as well for the required amounts of water and cement the biocarbon water correction factor is directly used to calculate the composition of the concrete mixtures. In this regard it is possible to calculate the composition by starting from a given or targeted amount of cement or a given or targeted water amount for a reference concrete mixture.

A reference concrete mixture is herein understood as a concrete mixture without the addition of biocarbon, which has targeted properties, that the concrete mixture optimized according to the method of the invention should also fulfill or is intended for despite using biocarbon.

In an embodiment the required total amount of water is determined as sum of a water content added during pre-wetting the biocarbon and an added water amount during mixing the concrete mixture.

According to an aspect of the present disclosure the biocarbon material can be pre-wetted and thus saturated or partially saturated with the water added to the biocarbon in advance of addition of the biocarbon to the concrete mixture. Preferably the water is applied to the biocarbon in advance of its addition to the concrete mixture and with sufficient time to reach partial saturation or saturation equilibrium. Mechanical mixing or temperature controlling can be used to accelerate the process of saturation. This is to provide a homogeneous distribution and absorption throughout the micro and macro pore structures of the biocarbon material. Additional water can be added during mixing on top of the amount of water used during pre-wetting.

An aspect of the present disclosure is that a pre-wetting of the biocarbon to complete biocarbon water pre-saturation before concrete mix integration is beneficial for strength development, workability and/or consistency. Due to practical constraints, it may not be feasible to fully pre-saturate the biocarbon materials in advance of concrete integration. Therefore, partial pre-saturation is a beneficial alternative. A correlation has been identified by the present disclosure, between the biocarbon material characteristics and the percentage of the water pre-wetted to the biocarbon. By using this technology, the biocarbon types are pre-wetted with a percentage of the mixing water prior to integration.

The percentage of the water pre-wetted can be optimized to provide good fresh concrete workability and structural strength.

The order of addition comprises preferably in the mentioned range the following steps: blending fine and coarse aggregates, adding 100% of the selected at least one biocarbon to the fine and coarse aggregates, adding at least the amount of water according to the biocarbon water correction factor to the mix of at least one biocarbon and aggregates, adding cement, adding water up to the total amount of water, optionally adding at least one plasticizer, preferably at least one superplasticizer.

According to a further embodiment determining the set of parameters comprises using at least one further material property of the at least one biocarbon, the at least one further material property comprise at least one of
carbon content [%] measured according to DIN 51732,
ash content [%] measured according to DIN 51719,
oxide content,
random reflectance,
surface morphology and surface charge,
water content [%] according to EN 1097-5,
particle size distribution measured according to ISO 13320:2020 or via a sieving analysis (EN 933-1 and optional ISO 9276-2:2014 for visualization),
median particle size,
particle size and shape measured according to ISO 19749:2021 and
specific surface area [m2/g] measured according to ISO 9277:2014.

Especially the mentioned material properties of the at least one biocarbon influence the properties and performance of the later concrete mixture and in parts also the biocarbon water correction factor. Thus it is beneficial to take those material properties and especially their relationships with performance or processing parameters of concrete mixtures into account, when determining parameters for the processing of the at least one biocarbon and/or the concrete mixture and especially in controlling the processing based on these properties. It is preferred if the further material properties are controlled as part of the method based on the set of parameters.

Further material properties, which can beneficially be taken into account in determining the set of parameters:
Biocarbon feedstock [source of the biomass material, e.g. corn stover, beech woodchips etc.]
Thermal conversion process (as described in Table 1) including but not limited to feedstock residence time, average temperature, temperature distribution, heating and cooling rates, reactor atmosphere including oxygen content and availability.
Bulk density [kg/m3] measured according to VDLUFA method A 13.2.1 or ISO 17828:2015 or EN 1097-3
Specific density [g/cm3] measured according to DIN 66137 or ISO 12154:2014
Mohs hardness [Mohs scale value] measured according to for example ISO 6769:2022
Porosity measured according to ISO 15901-2:2006
Particle characteristic shape measured according to ISO 19749:2021
Water holding capacity [%] measured according to ISO 1018:2019 or ISO 11274:2019 or ISO 14238:2014-03
Moisture content [%] according to DIN EN ISO 18134-1:2023-01
Water saturation rate measured according to ISO 62:2008 or ISO 20158:2018
Auto-ignition temperature [° C.] measured according to ISO/TS 21911-2:2022 or ISO 871:2006
Minimum ignition energy [mJ] measured according to ISO/IEC 80079-20-2:2016 or EN 13821 or ASTM E2019
Min ignition temperature (dust/air mix) [° C.] measured according to EN ISO/IEC 80079-20-2:2016
Kst value measured according to EN 14034 or ASTM E1226-10 or ISO 6184-1:1985
Conductivity [mS/cm] measured according to ISO 14309:2019 or ISO 11265
Zeta potential [mV] measured according to the guidelines of ISO/TR 19997:2018
Lower explosion limit [%] measured according to EN 14034 or ASTM E1226-10 or ISO 6184-1:1985
Total carbon content [%] measured according to DIN 51732 or ISO 16948:2015
Org. carbon content [%] measured according to DIN 51732 or ISO 16948:2015
Hydrogen content [%] measured according to DIN 51732 or ISO 16948:2015
Total nitrogen content [%] measured according to DIN 51732 or ISO 16948:2015
Sulphur content [%] measured according to DIN 51724-3:2012 or ISO 16994:201
Oxygen content [%] measurement according to DIN 51733 or ISO 1928:2020 or ISO 18125:2017
Total inorganic carbon [%] measured according to DIN 51732 or ISO 16948:2015
Carbonate-CO₂ [%] measured according to DIN 51726 or measured according to DIN 51732 or ISO 16948:2015
Hydrogen-carbon ratio measured according to DIN 51732 or ISO 16948:2015
Hydrogen-org. carbon ratio measured according to DIN 51732 or ISO 16948:2015
Oxygen-carbon ratio measurement according to DIN 51733 or ISO 1928:2020 or ISO 18125:2017
Volatile Matter Content [%] measured according to ISO 18123:2023
pH in CaCl₂) measured according to DIN EN ISO 10390:2022-08
Salt content [g/kg] measured according to BGK III. C2: 2006-09
HCl in insoluble ash [%] measured according to VDLUFA III 8.2 or ISO 776:2011
Calcium as CaO [% of ash] measured according to DIN EN ISO 11885 (E22):2009-09
Iron as Fe2O3 [% of ash] measured according to DIN EN ISO 11885 (E22): 2009
Potassium as K2O [% of ash] measured according to DIN EN ISO 11885 (E22): 2009-09
Magnesium as MgO [% of ash] measured according to DIN EN ISO 11885 (E22): 2009-09
Sodium as Na2O [% of ash] measured according to DIN EN ISO 11885 (E22): 2009-09
Phosphorus as P2O5 [% of ash] measured according to DIN EN ISO 11885 (E22): 2009-09
Sulphur as SO3 [% of ash] measured according to DIN EN ISO 11885 (E22): 2009-09
Silicon as SiO2 [% of ash] measured according to DIN EN ISO 11885 (E22): 2009-09 Water-soluble chloride ion [%] measured according to EN 1744-1
Acid-soluble sulphate [%] measured according to EN 1744-1
Total sulphur content [%] measured according to EN 1744-1
Fire resistance according to EN 13501-1
Trace elements content (inc. heavy metals) [mg/kg] measured according to measured according to DIN EN ISO 11885 (E22): 2009-09
PCB content [μg/kg] measured according to measured according to DIN EN ISO 11885 (E22): 2009-09
Polychlorinated dibenzo-p-dioxins and furans content [ng/kg] measured according to DIN 38414-S24: 2000-10 or ISO 13914:2023
Sum 8 PAH [mg/kg] measured according to DIN ISO 13877:2000
Sum 16 PAH [mg/kg] measured according to DIN ISO 13877:2000

According to an embodiment determining the set of parameters additionally comprises using at least one performance parameter of at least one known concrete mixture and/or an influence of at least one biocarbon with known material properties on this performance parameter, wherein the at least one performance parameter comprises
mechanical strength performance comprising 24 h, 7 and/or 28 day compressive and/or flexural strength and/or
concrete rheological performance comprising workability and/or flowability and/or
slump retention and/or
flow diameter retention and/or
thermal conductivity and/or
fresh density and/or
dry density and/or
setting time and/or
capillary porosity and/or
permeability and/or
long-term stability and/or
I carbonation and/or
freeze thaw resistance and/or
air content and/or
fire/heat resistance and/or
shrinkage.

An optimization of a concrete mixture with regard to these performance parameters is beneficial for several use cases and can be achieved through determination of the set of parameters for processing based on known relationships between properties of biocarbons and concrete mixtures and subsequently controlling the processing based on these relationships. Especially those performance parameters are positively influenced when the biocarbon water correction factor is used in for the preparation of the concrete mixture. For example it is shown in FIG. 19 that with biocarbon water correction factor in the range of 50 to 100% favorable 28 day compressive strength values can be reached for different biocarbon dosages.

In an embodiment in determining the set of parameters additionally at least one relationship between a mechanical strength of a concrete mixture and at least one of the following material properties of biocarbon: carbon content, ash content, oxide content, surface morphology and surface charge, specific surface area, particle size distribution, water content of the biocarbon, random reflectance, water absorption and water holding capacity feedstock material, pH (at integration and variability over time) and average feedstock processing temperature is taken into account. Thus influences of these properties beside their influence via the water absorption and thus via the biocarbon water correction factor on a mechanical strength can be taken into account. There exist numerous relationships between these properties and a mechanical strength, e.g. the mechanical strength after 28 day. In the following disclosure if not stated otherwise when 28 d strength is mentioned, this means the 28 day compressive strength. It is preferred, if the mentioned relationships are used in determining the set of parameters and thus controlling of the processing of the at least one biocarbon and/or the concrete mixture is based on these relationsships and especially comprises controlling the at least one of the material properties.

Mechanical strength performance is one of the concrete performance indicators provided by the present disclosure. Biocarbon(s) can be integrated into a standard mixture design and assessed according to common norm testing protocols (e.g. EN 196, EN 12350, EN 12390).

A biocarbon material of the present disclosure can fall into the following classes Class A—wood based; Class B—Food/Crop/Agriculture; Class C—Sewages and other based on their feedstock material. Examples of feedstock sources include, but are not limited to:

### Class A: Woody and Wood-Like Residues

Woodchips (across multiple wood material sources)
Green wood waste
Waste wood residues (inc. stumps, branches, leaves)
Bamboo
   Class B: Biomass Processing Residues (e.g. As Part of Food Prep and Production)
Cocoa shells, husks, and skins
Exotic fruit residues
Olive pits
Grapevine residues
Rice husks
Palm fruit residue
Shea nut residue
Grain husks
Hemp residue
Sunflower seed husks
Herb residues

### Class B: Agricultural Residues

Straw
Corn stalks
Corn husks
Corn cob

### Class C: Manures and Sludges

Manure
Sewage sludge
Digestate residue

Non-limiting examples of biocarbons according to the present disclosure are shown in the following Table.

TABLE 3
Biocarbon type characterization examples

| | |
|---|---|
| Material name: | Biocarbon blend(s) |
| Intended use: | Concrete ingredient replacer |
| Color: | Black |
| Odor: | Neutral |

| Class | A | B | C | |
|---|---|---|---|---|
| Ash/carbon | ≤0.01-0.15 | >0.15-0.5 | >0.5 | DIN 51719 or ISO |
| ratio range | | | | 18122: 2022/DIN 51732 |
| | | | | or ISO 16948: 2015 |
| Specific | 60-470 | 10-400 | 50-170 | ISO 9277: 2014 |
| Surface Area | | | | |
| (m2/g) | | | | |
| Bulk Density <3 | 145-404 | 32-420 | 200-700 | VDLUFA method A |
| mm (kg/m3): | | | | 13.2.1 or ISO |
| | | | | 17828: 2015 |
| Water holding | 150-455 | 85-355 | 85-240 | ISO 1018: 2019 or ISO |
| capacity (%) | | | | 11274: 2019 or |
| Carbon content | 75-99 | 60-85 | 25-60 | DIN 51732 or ISO |
| (% dry basis): | | | | 16948: 2015 |
| Nitrogen | 0.15-7 | 0.5-16.50 | 0.7-2.7 | DIN 51732 or ISO |
| content (%): | | | | 16948: 2015 |
| Hydrogen | 0.2-3.7 | 0.1-6 | 0.1-1.2 | DIN 51732 or ISO |
| content(%): | | | | 16948: 2015 |
| Ash Content | 2-12 | 18-33 | 33-75 | DIN 51719 or ISO |
| (%): | | | | 18122: 2022 |

In an embodiment the set of parameters is determined taking into account that a water content of the biocarbon before adding to the concrete mixture of 10 to 100% of the water absorption of the biocarbon improves a 28 d strength for all concrete mixtures. In FIG. 20a for example for biocarbons of class A and B in a flowable concrete mixture and for several water to cement ratios w/c a 28 d compressive strength is shown depending on the amount of water added to the biocarbon during pre-wetting as percentage of the absorption of the biocarbon. FIG. 20b the same relation is shown for biocarbons of class A and B in an earth moist concrete.

In particular, it is beneficial, when in determining the set of parameters it is additionally taken into account that for a flowable concrete a 28 d strength is optimized with a water content of the biocarbon before adding to the concrete mixture of 10 to 100% of the water absorption, wherein the water content is preferably in the range 40 to 70% of the water absorption of the biocarbon, wherein the 28 d strength is preferably at least in strength class C25/30 and/or C30/37.

Thus in determining the set of parameter the recognition that preferred 28 d strength is achieved with the water content in these ranges in taken into account when determining for example targeted water content, median particle size and other targeted properties.

It is furthermore beneficial when in determining the set of parameters it is additionally taken into account that for an earth moist concrete a 28 d strength is optimized with a water content of the biocarbon before adding to the concrete mixture of 10 to 100% of the water absorption, wherein the water content is preferably in the range 20-50% of the water absorption of the biocarbon, wherein the 28 d strength is preferably at least 15 MPa and wherein for a lightweight earth moist concrete the 28 d strength is preferably at least 1.5 MPa. As used in the present disclosure “lightweight earth moist concrete” refers to earth moist concrete having a density of ≤2000 kg/m3.

It is furthermore beneficial when in determining the set of parameters it is additionally taken into account that a 28 d strength is optimized with a median particle size range of 0.001 mm to 0.500 mm, for a biocarbon dose up to 180 kg/m³, wherein for a flowable concrete the 28 d strength is preferably optimized with a median particle size range of 0.001 to 0.030 mm and/or with a median particle size of 0.010 mm, in particular containing 99% of particles finer than 0.100 mm, 95% of particles finer than 0.063 mm, 70% of particles finer than 0.025 mm, wherein the 28 d strength for a flowable concrete is preferably at least 30 MPa, and/or wherein for an earth moist concrete a 28 d strength is preferably optimized with a median particle size range of 0.001 to 0.080 mm and/or with a median particle size of 0.046 mm, in particular containing 99% of particles finer than 2.000 mm, 97% of particles finer than 1.000 mm, 87% of particles finer than 0.500 mm, 72% of particles finer than 0.200 mm, 64% of particles finer than 0.100 mm, 58% of particles finer than 0.063 mm, 32% of particles finer than 0.025 mm, wherein the 28 d strength for an earth moist concrete is preferably at least 15 MPa, and/or wherein for a lightweight earth moist concrete a 28 d strength is preferably optimized with a median particle size range of 0.100 to 0.500 mm and/or a median particle size of 0.380 mm, in particular containing 99% of particles finer than 4.000 mm, 86% of particles finer than 2.000 mm, 76% of particles finer than 1.000 mm, 58% of particles finer than 0.500 mm, 32% of particles finer than 0.200 mm, 13% of particles finer than 0.100 mm, wherein the 28 d strength is for a lightweight earth moist concrete preferably at least 1.5 MPa. In FIG. 21 the 28 d compressive strength is shown depending on the median particle size d50 of the used biocarbon for a flowable concrete. The highest strengths are achieved for a d50 of 0.01 mm.

It is furthermore beneficial when in determining the set of parameters it is additionally taken into account a 28d strength is optimized if at least one additive, comprising preferably a plasticizer, more preferred a superplasticizer is present in the concrete mixture according to an additive optimization parameter

$${R}_{AD} = \frac{B W C P \cdot {c}_{AD}}{{d}_{biocarbon}} ,$$

wherein BWCP is the biocarbon water correction percentage, c_{AD} is a dose of additive in weight-% of a cement content, d_{biocarbon} is the dosage of biocarbon in kg/m³ and RAD is in the range of 2.0 to 0.1, preferably 1.9 to 0.6, preferably between 1.2 and 0.78, more preferably between 1.08 and 0.78.

It is furthermore beneficial when in determining the set of parameters it is additionally taken into account that a 28 d strength is optimized if at least one additive, comprising preferably a plasticizer, more preferred a superplasticizer, is present in the concrete mixture according to an additive w/c optimization parameter

$${X}_{ADwc} = \frac{{( \frac{w}{c} )}_{corr} \cdot {c}_{AD}}{{d}_{biocarbon}} * 100 , wherein {( \frac{w}{c} )}_{corr}$$

is the corrected water-cement ratio, c_{AD} is a dose of additive in weight-% of a cement content, d_{biocarbon} is the dosage of biocarbon in kg/m³ and X_{ADwc} is in the range of 2.50 to 0.30, preferably between 2.0 and 0.4, more preferably between 1.44 and 0.45. The use of additives, especially superplasticizers according to the additive w/c optimization parameter allows for further enhancement of concrete mixtures in view of workability and performance.

X_{ADwc} is especially preferred for flowable concrete. X_{ADwc} is preferably used for concrete with a biocarbon dosage of below 180 kg/m³ biocarbon, preferably used for concrete with a dosage of below 150 kg/m³ biocarbon, preferably used for concrete with a dosage of below 140 kg/m³ biocarbon, preferably used for concrete of below 130 kg/m³ biocarbon, preferably used for concrete of below 120 kg/m³.

Table 4 shows values for R_{AD} and X_{ADwc} for a exemplary concrete mixtures. The optimized strength values are within a similar range the one from a reference concrete mixture, which did not comprise biocarbon. Cement type used in these examples was CEM I 42.5R (supplied from Spenner)_(also reproducible with a CEM I 42.5R (supplied from Cemex)), Su-perplastisizer was Master Glenium ACE 430 (supplied from Master Builder Solution GmbH, being a Polycarboxylate Ether), biocarbon was a class A Biocarbon. Similar results were obtained with a class B biocarbon and by using CEM I 42.5 R (supplied from Cemex).

TABLE 4

| | Dosage Bio- | | | | R_AD = | X_ADwc = | |
|---|---|---|---|---|---|---|---|
| BWCP | carbon | SP | SP | | BWCP * | (w/c) * AD/ | Strength |
| [%] | [kg/m³] | ID | [% bwoc] | wₜₒₜₐₗ/c | AD/BD | BD *100 | [MPa] |
| 50 | 60 | ACE 430 | 1.50% | 0.583 | 1.250 | 1.458 | 30 |
| 57 | 60 | ACE 430 | 1.00% | 0.603 | 0.950 | 1.005 | 35 |
| 100 | 60 | ACE 430 | 0.30% | 0.725 | 0.500 | 0.363 | 24 |
| 30 | 80 | ACE 430 | 2.91% | 0.722 | 1.091 | 2.626 | 18 |
| 60 | 80 | ACE 430 | 1.32% | 0.800 | 0.990 | 1.320 | 27 |
| 100 | 80 | ACE 430 | 0.53% | 0.942 | 0.663 | 0.6232 | 22 |

It is furthermore beneficial when in determining the set of parameters it is additionally taken into account that a 28 d strength is optimized for an earth moist concrete if water, cement and the at least one biocarbon are present in the concrete mixture according to a biocarbon w/c optimization parameter

$${Y}_{Bwc} = \frac{{( \frac{w}{c} )}_{corr}}{{d}_{biocarbon}} * 100 , wherein {( \frac{w}{c} )}_{corr}$$

is the corrected water-cement ratio and d_{biocarbon} is the dosage of biocarbon in kg/m³ and Y_{Bwc} is in the range of 0.45 and 1.6, preferably between 0.6 and 1.2, more preferably between 0.7 and 1.0. A concrete mixture with this range of the biocarbon w/c optimization parameter shows good strength performance.

It is furthermore beneficial when in determining the set of parameters it is additionally taken into account that for a flowable concrete a 28 d strength is improved using a corrected water-cement ratio between 0.40 and 0.70, preferably 0.45 to 0.68 and for an earth moist concrete a 28d strength is improved using a corrected water-cement ratio between 0.36 and 0.80, preferably 0.40 to 0.65.

For an exemplary biocarbon the 28 d compressive strength of a flowable concrete is shown in FIG. 22 depending on the corrected water-cement ratio. It can be seen, that over the whole range of 0.40 to 0.70 the 28 d strength is not compromised, while being slightly better between 0.45 and 0.68.

In an embodiment in determining the set of parameters additionally at least one relationship between a rheological performance and at least one of the following material properties of biocarbons: water content, water absorption, water holding capacity, median particle size, particle size distribution, particle size and shape, specific surface area, ash content, carbon content and/or at least one of the following concrete composition parameters: pH (at integration and fluctuations over time) and zeta potential is taken into account. Preferably it is taken into account that for a median particle size in a range of 0.001 to 0.35 mm the flow diameter is in a range of 300 to 600 mm. It is preferred, if the mentioned relationships are used in determining the set of parameters and thus controlling of the processing of the at least one biocarbon and/or the concrete mixture is based on these relationsships and especially comprises controlling the at least one of the material properties accordingly.

It is furthermore beneficial when determining the set of parameters additionally comprises using that with a water content of the biocarbon before adding to the concrete mixture of 25-75% of the water absorption a slump retention and/or flow diameter retention of up to 45 min is maintained.

FIGS. 23 and 24 show the evolution of the average flow diameter over time for concrete mixtures with a biocarbon of class A (FIG. 23) and class B (FIG. 24) and different water contents of the respective biocarbon at a biocarbon dosage of 60 kg/m³. The average flow diameter is kept constant over a time of up to 45 min for both biocarbon classes especially with water content in the range of 25-75% of the water absorption.

Other relations that can be used in determining the set of parameters is that a higher water holding capacity or water absorption of a biocarbon leads to a reduction in the flow diameter of concrete and/or that the finer a median particle size the higher is the flow diameter of mortar or concrete, wherein preferably the flow diameter is in a range of 300 to 600 mm, more preferred 350 to 550 mm, for a median particle size of range 0.001 to 0.350 mm, wherein preferably the median particle size is 0.010 mm, 0.046 mm or 0.090 mm.

FIG. 25 shows as an example of the flow diameter in relation to the median particle size d50 for a flowable concrete. The flow diameter is the highest for a median particle size of 0.010 mm.

In a further embodiment determining the set of parameters additionally comprises using that for a targeted slump retention and/or flow diameter retention of 0 to 30 min a polycarboxylate PCE based superplasticizer is favorable, preferably in a dose of 0.2 to 3.0%, more preferred 0.4 to 1.2%, and for targeted slump retention and/or flow diameter retention of 30 to 60 min a lignosulfonate based superplasticizer is favorable, preferably in a dose of 0.2 to 1.5%, more preferred 0.3 to 1.1%.

FIG. 26 shows the flow diameters 4 min and 90 min after mixing for a reference mixture without biocarbon and for a mixture with 2% biocarbon with different superplasticizers. All shown superplasticizer lead to good performance regarding the flow diameter retention with the lignosulfonate showing a tendency to larger retention time.

In a further embodiment in determining the set of parameters additionally at least one relationship between thermal conductivity of the concrete mixture and at least one of the following material properties of biocarbons: specific density, specific surface area, carbon content, particle density, bulk density, particle size distribution, median particle size and ash content is taken into account, preferably wherein it is taken into account that the thermal conductivity is lower with a higher particle density. It is preferred, if the mentioned relationships are used in determining the set of parameters and thus controlling of the processing of the at least one biocarbon and/or the concrete mixture is based on these relationships and especially comprises controlling the at least one of the material properties.

It is furthermore beneficial, if at least one relationship between CO₂ performance and at least one of the following material properties of biocarbons or concrete composition parameters: carbon content, silica content, and ash content is taken into account in determining the set of parameters.

In a further embodiment in determining the set of parameters at least one relationship between fresh density of a concrete mixture and at least one of the following material properties of biocarbons: particle density, specific density, bulk density, particle size distribution, median particle size, water absorption, water holding capacity, specific surface area and median particle size and/or the corrected water-cement ratio of concrete mixtures are taken into account, wherein in particular it is taken into account that the fresh density is higher the lower the median particle size is. In particular it can be taken into account that for a median particle size of range 0.001 to 0.050 mm and/or for a median particle size of 0.010 mm or 0.046 mm the fresh density is in a range of 2100 to 2350 kg/m³ and/or that the fresh density is increased for a flowable concrete for a corrected water-cement ratio between 0.40-0.70, preferably 0.45 to 0.68. It is preferred, if the mentioned relationships are used in determining the set of parameters and thus controlling of the processing of the at least one biocarbon and/or the concrete mixture is based on these relationsships and especially comprises controlling the at least one of the material properties.

FIG. 27 shows that the fresh density is kept constant for a flowable concrete for a corrected water-cement ratio between 0.550 and 0.70. FIG. 28a and FIG. 28b show the highest fresh density for a biocarbon of class A with biocarbon dosage of 60 kg/m³ with a median particle size of 0.01 mm for both, flowable concrete (FIG. 28a) and earth moist concrete (FIG. 28b).

In an embodiment in determining the set of parameters at least one relationship between dry density of the concrete mixture and the median particle size of the biocarbon is taken into account, in particular wherein it is taken into account that the dry density is higher the lower the median particle size is, wherein in particular for a median particle size of range 0.001 to 0.050 mm and/or for a median particle size of 0.010 mm or 0.046 mm the dry density is in a range of 2100 to 2350 kg/m³.

FIG. 29 shows the highest dry density for a biocarbon of class A with biocarbon dosage of 60 kg/m³ with a median particle size of 0.01 mm for an earth moist concrete.

In an embodiment in determining the set of parameters at least one relationship between setting time and the median particle size is taken into account, in particular wherein it is taken into account that the setting time is lower the lower the median particle size is, wherein the setting time measured according to EN 196-3.

In a further embodiment the at least one biocarbon is a mixture of at least two biocarbons. Mixtures of at least two biocarbon allow to use favorable properties of both, e.g. adjusting a particle size distribution by using two biocarbons with different particle size distributions or adjusting the ash content.

In a preferred embodiment the method further comprises at least one step of processing the at least one biocarbon and/or the concrete mixture using the set of parameters, in particular at least one of the following processing steps:
selecting at least one biocarbon of the group of possible biocarbons for adding into the concrete mixture and/or
treating the at least one biocarbon preferably via at least pre-wetting and/or adjustment of the median particle size and/or
dosing the at least one biocarbon and/or
adding water and/or
adding cement and/or
adding at least one additive, in particular at least one plasticizer, in particular at least one superplasticizer.

Thus the at least one biocarbon and/or the concrete mixture are processed with the set of parameters leading to an concrete mixture with enhanced performance according to the set of parameters determined via the preferably computer-implemented method.

Thereby in the at least one step of processing the set of parameters provided preferably by the computer-implemented method can be used to control processing devices able and arranged to conduct the respective at least one step of processing.

It is thereby preferred if water and/or cement is added into the concrete mixture based on the biocarbon water correction factor, preferably with the biocarbon water correction factor being in the range of 10 to 100% of the water absorption, more preferred 40-70% of the water absorption of the at least one biocarbon for flowable concrete mixture or 20-50% of the water absorption for an earth moist concrete mixture. The addition of water and/or cement according to the biocarbon water correction factor allows the maintainance and further improvement of especially the strength but also the rheological performance of concrete mixtures in ranges, which are reached without biocarbon, despite the addition of biocarbon. Thus this leads to concrete mixtures with a largely improved long-term CO₂ equivalent storage potential and overall CO2 footprint reduction with high mechanical strength and also good rheological performance. In other words with the consideration of the biocarbon water correction factor for the addition of water and/or cement strength of a concrete mixture is improved compared to concrete mixture with added biocarbon but without observance of the biocarbon water correction factor.

It is further preferred, when water and/or cement is added into the concrete mixture according to the corrected water-cement ratio being for a flowable concrete between 0.40 and 0.70, preferably 0.45 to 0.68 and for an earth moist concrete being between 0.36 and 0.80, preferably 0.40 to 0.65. With a corrected water-cement ratio in the given ranges, especially the strength performance of the concrete mixture is enhanced.

It is further preferred, when for an earth moist concrete water, cement and the at least one biocarbon are added in the concrete mixture according to the biocarbon w/c optimization parameter

$${Y}_{Bwc} = \frac{{( \frac{w}{c} )}_{corr}}{{d}_{biocarbon}} * 100 , wherein {( \frac{w}{c} )}_{corr}$$

is the corrected water-cement ratio and d_{biocarbon} is the dosage of biocarbon in kg/m³ and Y_{Bwc} is in the range of 0.45 and 1.6, preferably between 0.6 and 1.2, more preferably between 0.7 and 1.0.

In an embodiment water and/or cement is added into the concrete mixture according to the corrected water-cement ratio

$${( \frac{w}{c} )}_{corr} = {( \frac{w}{c} )}_{t} \cdot ( 1 + \frac{B W C F \cdot {m}_{biocarbon}}{{w}_{t}} ) ,$$

wherein BWCF is the biocarbon water correction factor,

$${( \frac{w}{c} )}_{t}$$

is a targeted water-cement ratio for a reference concrete mixture without biocarbon, m_{biocarbon} is the mass of biocarbon in the concrete mixture and wₜ is a targeted water amount for the reference concrete mixture without biocarbon and/or according to

$${( \frac{w}{c} )}_{corr} = {( \frac{w}{c} )}_{t} \cdot ( 1 + \frac{B W C F \cdot {m}_{biocarbon}}{{{c}_{t} ( \frac{w}{c} )}_{t}} ) ,$$

wherein BWCF is the biocarbon water correction factor,

$${( \frac{w}{c} )}_{t}$$

is a targeted water-cement ratio for a reference concrete mixture without biocarbon, m_{biocarbon} is the mass of biocarbon in the concrete mixture and cₜ is a targeted cement amount for the reference concrete mixture without biocarbon and/or according to wₜₒₜₐₗ=wₜ+BWCF·m_{biocarbon}, wherein BWCF is the biocarbon water correction factor, wₜ is a targeted water amount according to the targeted water-cement ratio for the reference concrete mixture without biocarbon

$${( \frac{w}{c} )}_{t}$$

and m_{biocarbon} is the mass of biocarbon in the concrete mixture and/or according to

$$c = \frac{( {w}_{t} + BWCF \cdot {m}_{biocarbon} )}{{( \frac{w}{c} )}_{corr}} ,$$

wherein BWCF is the biocarbon water correction factor,

$${( \frac{w}{c} )}_{corr}$$

is the corrected water-cement ratio, m_{biocarbon} is the mass of biocarbon in the concrete mixture and wₜ is a targeted water amount for the reference concrete mixture without biocarbon.

These formulas allow for an improved concrete mixture based on the biocarbon water correction factor.

In an embodiment the required total amount of water is a sum of a water content added during pre-wetting the biocarbon and an added water amount during mixing the concrete mixture. As discussed previously water can be added in a pre-wetting step of the biocarbon but also during mixing the concrete mixture. It is preferred if at least a part of the required total amount of water is added during pre-wetting. It is preferred, if the at least one biocarbon is pre-wetted to a water content of 10-100% of the water absorption of the at least one biocarbon, preferably pre-wetted to a water content in the range of 10-75% of the water absorption, more preferred in the range of 10-50% of the water absorption, even more preferred in the range of 10 to 25% of the water absorption of the at least one biocarbon for a flowable concrete or to a water content of 10 to 80% of the water absorption, more preferred in the range of 10 to 60% of the water absorption, even more preferred in the range of 40 to 60% of the water absorption of the at least one biocarbon for an earth moist concrete. Especially these ranges for the pre-wetting enhance the workability of the biocarbon in the concrete mixture and also the workability of the concrete mixture as a whole.

It is preferred, if the median particle size range of the at least one biocarbon is adjusted to a range of 0.001 mm to 0.5 mm. The median particle size range is adjusted for a flowable concrete mixture to a median particle size range of 0.001 to 0.030 mm and/or to a median particle size of 0.010 mm, in particular containing 99% of particles finer than 0.100 mm, 95% of particles finer than 0.063 mm, 70% of particles finer than 0.025 mm. It is further preferred, if the median particle size range for an earth moist concrete mixture is adjusted to a median particle size range of 0.001 to 0.080 mm and/or to a median particle size of 0.046 mm, in particular containing 99% of particles finer than 2.000 mm, 97% of particles finer than 1.000 mm, 87% of particles finer than 0.500 mm, 72% of particles finer than 0.200 mm, 64% of particles finer than 0.100 mm, 58% of particles finer than 0.063 mm, 32% of particles finer than 0.025 mm. If is further preferred, if the median particle size range is adjusted for a lightweight earth moist concrete mixture to a median particle size range of 0.100 to 0.500 mm and/or to a median particle size of 0.380 mm, in particular containing 99% of particles finer than 4.000 mm, 86% of particles finer than 2.000 mm, 76% of particles finer than 1.000 mm, 58% of particles finer than 0.500 mm, 32% of particles finer than 0.200 mm, 13% of particles finer than 0.100 mm.

In an embodiment the order of addition is blending fine and coarse aggregates, adding 100% of the at least one biocarbon to the fine and coarse aggregates, adding at least the amount of water according to the biocarbon water correction factor to the mix of at least one biocarbon and aggregates, adding cement, adding water up to the total amount of water, optionally adding at least one plasticizer, preferably at least one superplasticizer.

In a further embodiment at least one additive, comprising preferably a plasticizer, more preferred a superplasticizer, is added into the concrete mixture according to the additive w/c optimization parameter

$${X}_{ADwc} = \frac{{( \frac{w}{c} )}_{corr} \cdot {c}_{AD}}{{a}_{biocarbon}} * 100 , wherein {( \frac{w}{c} )}_{corr}$$

is the corrected wa-ter-cement ratio, c_{AD} is a dose of additive in weight-% of a cement content, d_{biocarbon} is the dosage of biocarbon in kg/m³, X_{ADwc} being in the range of 2.50 to 0.30, preferably between 2.0 and 0.4, more preferably between 1.44 and 0.45.

It is further preferred if a polycarboxylate (PCE) based superplasticizer, preferably in a dose of 0.2 to 3.0%, more preferred 0.4 to 1.2%, is added to the concrete mixture and/or wherein a lignosulfonate based superplasticizer preferably in a range of 0.2 to 1.5%, more preferred 0.3 to 1.1%, is added to the concrete mixture. While polycarboxylate based superplasticizer enhance slump retention in the range of 0 to 30 min, for targeted slump retention of 30 to 60 min lignosulfonate based superplasticizers are preferred.

According to a second aspect the invention relates to a concrete mixture or an intermediate product, in particular a customized biocarbon, produced or processed according to the method of the first aspect of the invention, especially according to the method comprising steps of processing the at least one biocarbon and/or the concrete mixture. A customized biocarbon is a biocarbon with beneficial material properties for the use in concrete mixtures and is accordingly processed.

According to a third aspect the invention relates to a customized biocarbon for use in flowable concrete, in particular processed according to the method of the first aspect of the invention, especially according to the method comprising steps of processing the at least one biocarbon and/or the concrete mixture, having
a water content of 10-100%, preferably of 40-70% of the water absorption of the biocarbon and
a median particle size range of 0.001 mm to 0.5 mm, preferably of 0.001 to 0.030 mm, preferably having a median particle size of 0.010 mm, in particular containing 99% of particles finer than 0.100 mm, 95% of particles finer than 0.063 mm, 70% of particles finer than 0.025 mm.

Such customized biocarbon allows for flowable concrete with good strength and workability despite the addition of biocarbon. Thus flowable concrete is with such a customized biocarbon enhanced regarding long-term CO₂ equivalent storage potential and overall CO₂ footprint reduction due to the addition of biocarbon while at the same time maintaining strength and workability.

According to a fourth aspect the invention relates to a customized biocarbon for use in earth moist concrete, in particular processed according to the method of the first aspect of the invention, especially according to the method comprising steps of processing the at least one biocarbon and/or the concrete mixture, having
a water content of 10-100%, preferably of 20-50% of the water absorption of the biocarbon and
a median particle size range of 0.001 mm to 0.5 mm, preferably of 0.001 to 0.080 mm, preferably having a median particle size of 0.046 mm, in particular containing 99% of particles finer than 2.000 mm, 97% of particles finer than 1.000 mm, 87% of particles finer than 0.500 mm, 72% of particles finer than 0.200 mm, 64% of particles finer than 0.100 mm, 58% of particles finer than 0.063 mm, 32% of particles finer than 0.025 mm.

Such customized biocarbon allows for earth moist concrete with good strength and consistency despite the addition of biocarbon. Thus earth moist concrete is enhanced regarding long-term CO₂ equivalent storage potential and overall CO₂ footprint reduction due to the addition of the customized biocarbon while at the same time maintaining strength and consistency.

According to a fifth aspect the invention relates to a customized biocarbon for use in lightweight earth moist concrete, in particular processed according to the method of the first aspect of the invention, especially according to the method comprising steps of processing the at least one biocarbon and/or the concrete mixture, having
a water content of 10-100%, preferably of 20-50% of the water absorption of the biocarbon and
a median particle size range of 0.001 mm to 0.5 mm, preferably of 0.100 to 0.500 mm, preferably a median particle size of 0.38 mm, in particular containing 99% of particles finer than 4.000 mm, 86% of particles finer than 2.000 mm, 76% of particles finer than 1.000 mm, 58% of particles finer than 0.500 mm, 32% of particles finer than 0.200 mm, 13% of particles finer than 0.100 mm.

Such customized biocarbon allows for lightweight earth moist concrete with good strength and consistency despite the addition of biocarbon. Thus lightweight concrete can be enhanced regarding CO₂ performance and low density due to the addition of the customized biocarbon while at the same time maintaining strength and consistency.

In a preferred embodiment the customized biocarbon is a mix of at least two biocarbons.

According to a sixth aspect the invention relates to a use of a biocarbon, in particular processed according to the method of the first aspect of the invention, especially according to the method comprising steps of processing the at least one biocarbon and/or the concrete mixture, in flowable concrete, the biocarbon having
a water content of 10-100%, preferably of 40-70% of the water absorption of the biocarbon and
a median particle size range of 0.001 mm to 0.5 mm, preferably of 0.001 to 0.030 mm preferably a median particle size of 0.010 mm containing 99% of particles finer than 0.100 mm, 95% of particles finer than 0.063 mm, 70% of particles finer than 0.025 mm.

According to a seventh aspect the invention relates to a use of a biocarbon, in particular produced according to the method of the first aspect of the invention, especially according to the method comprising steps of processing the at least one biocarbon and/or the concrete mixture, in an earth moist concrete, the biocarbon having
a water content of 10-100%, preferably of 20-50% of the water absorption of the biocarbon and
a median particle size range of 0.001 mm to 0.5 mm, preferably of 0.001 to 0.080 mm, preferably a median particle size of 0.046 mm, in particular containing 99% of particles finer than 2.000 mm, 97% of particles finer than 1.000 mm, 87% of particles finer than 0.500 mm, 72% of particles finer than 0.200 mm, 64% of particles finer than 0.100 mm, 58% of particles finer than 0.063 mm, 32% of particles finer than 0.025 mm.

According to an eighth aspect the invention relates to a use of a biocarbon, in particular processed according to the method of the first aspect of the invention, especially according to the method comprising steps of processing the at least one biocarbon and/or the concrete mixture, in lightweight earth moist concrete, the biocarbon having
a water content of 10-100%, preferably of 20-50% of the water absorption of the biocarbon and
a median particle size range of 0.001 mm to 0.5 mm, preferably of 0.100 to 0.500 mm, preferably a median particle size of 0.38 mm, in particular containing 99% of particles finer than 4.000 mm, 86% of particles finer than 2.000 mm, 76% of particles finer than 1.000 mm, 58% of particles finer than 0.500 mm, 32% of particles finer than 0.200 mm, 13% of particles finer than 0.100 mm.

According to a ninth aspect the invention relates to a concrete mixture comprising biocarbon, in particular produced according to the method of the first aspect of the invention, especially according to the method comprising steps of processing the at least one biocarbon and/or the concrete mixture, having a corrected water-cement ratio of

$${( \frac{w}{c} )}_{corr} = \frac{{w}_{t} + BWCF \cdot {m}_{biocarbon}}{c} ,$$

wherein BWCF is the biocarbon water correction factor, m_{biocarbon} is the mass of biocarbon in the concrete mixture c is the cement amount in the concrete mixture and wₜ is a targeted water amount for a reference concrete mixture without biocarbon and wherein preferably BWCF is in a range of 10-100% of the water absorption of the at least one biocarbon, more preferred of 40 to 70% of the water absorption, when the concrete mixture is a flowable concrete mixture, or of 20 to 50% of the water absorption, when the concrete mixture is an earth moist concrete mixture.

In an embodiment the biocarbon water correction percentage for a flowable concrete is in the range of 40 to 65%, more preferred 45 to 65%, more preferred 50 to 60%, more preferred 55 to 60%, more preferred 57 to 58%.

In a further embodiment the biocarbon water correction percentage for an earth moist concrete is in the range of 20 to 45%, more preferred 25 to 45%, more preferred 30 to 40%, more preferred 35 to 40%.

Concrete mixtures having such a corrected water-cement ratio and a water correction factor in the range of 10-100% are optimized regarding the long-term CO₂ equivalent storage potential and overall CO₂ footprint reduction, strength and workability and/or consistency.

In a preferred embodiment the concrete mixture comprises at least one additive, compriseing preferably a plasticizer, more preferred a superplasticizer according to the additive optimization parameter

$${R}_{AD} = \frac{BWCP \cdot {c}_{AD}}{{d}_{biocarbon}} ,$$

whrerein BWCP is the biocarbon water correction percentage, c_{AD} is a dose of additive in weight-% of a cement content, d_{biocarbon} is the dosage of biocarbon in kg/m³ and R_{AD} is in the range of 2.0 to 0.1, preferably 1.9 to 0.6, preferably between 1.2 and 0.78, more preferably between 1.08 and 0.78.

In a preferred embodiment the concrete mixture comprises at least one additive, comprissing preferably a plasticizer, more preferred a superplasticizer according to the additive w/c optimization parameter

$${X}_{ADwc} = \frac{{( \frac{w}{c} )}_{corr} \cdot {c}_{AD}}{{d}_{biocarbon}} * 100 , wherein {( \frac{w}{c} )}_{corr}$$

is the corrected water-cement ratio, c_{AD} is a dose of additive in weight-% of a cement content, d_{biocarbon} is the dosage of biocarbon in kg/m³ and X_{ADwc} is in the range of 2.50 to 0.30, preferably between 2.0 and 0.4, more preferably between 1.44 and 0.45. The use of additives, especially superplasticizers according to the additive w/c optimization parameter allows for further enhancement of concrete mixtures in view of workability and/or consistency and performance.

In a further preferred embodiment the concrete mixture comprises water, cement and the at least one biocarbon according to a biocarbon w/c optimization parameter

$${Y}_{Bwc} = \frac{{( \frac{w}{c} )}_{corr}}{{a}_{biocarbon}} * 100 , wherein {( \frac{w}{c} )}_{corr}$$

is the corrected water-cement ratio and d_{biocarbon} is the dosage of biocarbon in kg/m³ and Y_{Bwc} is in the range of 0.45 and 1.6, preferably between 0.6 and 1.2, more preferably between 0.7 and 1.0. The concrete mixture with this range of the biocarbon w/c optimization parameter shows good strength performance.

It is preferred if the corrected water-cement ratio is for a flowable concrete between 0.40 and 0.70, preferably 0.45 to 0.68 and is for an earth moist concrete between 0.36 and 0.80, preferably 0.40 to 0.65. With a corrected water-cement ratio in the given range, especially the strength performance of the concrete mixture is enhanced.

In a preferred embodiment, the concrete mixture, which is in particular a flowable concrete mixture, with a dosage of biocarbon of 60 kg/m³ to 180 kg/m³ has a 28 d compressive strength at least in strength class C25/30 and or C30/37 and/or a fresh density of at least 2100 kg/m³.

In a preferred embodiment, the concrete mixture, which is in particular an earth moist concrete mixture, with a dosage of biocarbon of 60 kg/m³ to 180 kg/m³ has a 28 d compressive strength of at least 15 MPa and/or a fresh density of at least 2100 kg/m³.

The use of the method according to the first aspect of the invention surprisingly allows high strength and fresh density of the final concrete mixture despite dosages of biocarbon in relatively high ranges. Thus the invention as a whole allows for concrete with relatively high strength and fresh density with considerably improved long-term CO2 equivalent storage potential and overall CO2 footprint reduction.

In the present disclosure, an example of a concrete mixture comprises the following ingredients or consists of the following components:
(a) cementitious based binder, preferably silicate cement, Portland cement, CEM I, CEM II, CEM III, CEM IV, CEM V, CEM VI and combinations thereof;
(b) one or more biocarbon materials;
(c) one or more aggregates, preferably selected from the group comprising, gravel, silt, sand, fines, light weight aggregates;
(d) optionally, one or more additives; and
(e) optionally, water.

In the manner described in the present disclosure, the binder fraction of the mixture can be replaced, between 1% to 30%, and/or the aggregate fraction can be replaced between 1% and 20% and/or other concrete ingredients replaced by between 1% and 50%.

Water can be adsorbed in any of the other components (a), (b), (c), and/or (d) (if present). Adsorption includes but is not limited to physical and chemical adsorption. The present disclosure includes pre-wetting (pre-saturation) of biocarbon as means for concrete integration. Preferably pre-wetting or pre-saturation (partial or full) of material (b) is defined based on the water absorption or water holding capacity as measured according to the aforementioned norms, as another example pre-wetting or pre-saturation (partial or full) of material (b) can also be defined based on the water content (wt. %) as measured according to the aforementioned norms.

Another aspect of the present disclosure is that the ingredient (b) is preferably not dry, when added to the concrete mixture. Not-dry means, that it comprises up to 100 wt. % of water of the material (b) water holding capacity, preferably 100 wt. % of water of the material (b) water holding capacity, in other words it is preferred that the water content of the at least one biocarbon before adding to the mixture is between 10 and 100% of the water absorption of the at least one biocarbon.

Typically, the ingredients (a), (c), and/or (d) are dry, as water starts the hydration reaction. Dry means, that it comprises less than 10 wt. % of water, more preferably less than 7.5 wt.-% of water, more preferably less than 5 wt.-% water, more preferably less than 4 wt.-% of water, more preferably less than 3 wt.-% of water, more preferably less than 2 wt.-% of water, more preferably less than 1 wt.-% of water, more preferably less than 0.75 wt.-% of water, more preferably less than 0.5 wt.-% of water, more preferably less than 0.25 wt.-% of water.

The skilled person is aware that the properties of a mixture are influenced by all components of the mixture. For example, one could not easily replace and/or remove the aggregates such as gravel, sand or silt from a concrete or mortar mixture while maintaining the same material properties. It was thus surprising that the use of a mixture as described herein allows concrete-material replacement without negatively influencing the properties, e.g. the compressive strength of the construction material while at the same time permitting the full CO₂ emissions of the material to be compensated for resulting in a CO₂ neutral concrete. The mixture may also include other additives.

Component (d) is/are selected from the group consisting of:
The one or more additives is/are selected from the group consisting of:
(i) (bio) polymers selected from the group comprising or consisting of cellulose and derivatives thereof, starch and derivatives thereof, lignin and derivatives thereof, preferably lignin sulfonates, kraft-lignins and lignin carboxylates, pectins and derivatives thereof, xanthan and derivates thereof, guarethers and derivatives thereof; chitin and derivatives thereof, algin and derivatives thereof, chitosan and derivatives thereof, cylcodextrins and derivatives thereof, dextrins and derivatives thereof; natural glues, hydrogel builders, plant lime, latex, rubber and derivatives thereof; proteins and peptides comprising one or more of the amino acids selected from the group comprising or consisting of alanin, glycin, lysin, asparagin, glutamin, glutamate and non-proteinogenic amino acids; industrial substances, residual polymeric substances and industrial by-products selected from the group comprising or consisting of industrial effluents, preferably corn steep liquor, lactose mother liquor, protein lysates and molasses, vegetable meals, preferably corn gluten meal, pea meal, fruit meals and protein wastes, preferably from yeast production, meat production, fruit production, vegetable production, egg production, dairy industry and papermaking; starch ethers, starch esters, starch carboxylates, cellulose esters, cellulose ethers, cellulose carboxylates, yeasts and derivatives or extracts thereof; liquid or dried polymer dispersions or polymers comprising organic acids, preferably sulfonic acids, carboxylic acids, peroxy carboxylic acids and thio carboxylic acids and salts thereof, sulfoxides, cyanates, thiocyanates, esters, ethers, thio ethers, oxides, thio oxides, amines, imines, hydrazines, hyrazones, amids, sulfates, nitriles, aldehydes, thio aldehydes, ketons, thioketons, oximes, alkohols, thiols, radicals, halogens, silanes, siloxanes, phosphates, phosponates, alkyls, allyls, aryls and derivatives thereof, wherein preferably the polymer(s) is/are biodegradable;
(ii) (poly) saccharides, extracellular substances and derivatives thereof selected from the group comprising or consisting of (poly) saccarides comprising lactose, glucose, fructose, saccharose and/or galactose, and microbial exopolysaccharides, preferably comprising lactose, saccharose, glucose, glucosamine, mannose, glycerin, gluconate, fructose and/or inulin;
(iii) organic acids and derivatives thereof, preferably selected from the group comprising or consisting of monocarboxylic acids, preferably formic acid, acetic acid, propionic acid, butyric acid, benzoic acid and salicylic acid, dicarboxylic acids, preferably oxalic acid, malonic acid, succinic acid, glutaric acid, adipic acid and maleic acid, fatty acids, keto acids, preferably pyruvic acid and acetoacetic acid, fruit acids, preferably malic acid and tartaric acid, hydroxy acids, preferably lactic acid, alpha hydroxy acids and beta hydroxy acids, tricarboxylic acids, preferably citric acid, more preferably carboxylates and esters of the before mentioned;
(iv) amino acids and derivatives thereof, preferably selected from the group comprising or consisting of alanin, glycine, lysine, glutamine, glutamate, and non-proteinogenic amino acids, preferably esters and amides thereof;
(v) substances changing the reaction mechanism, preferably retarders, accelerators, bleeding modifiers, hydrophilizers, hydrophobizers, air entraining agents, viscosity modifiers, expanders, accelerators, retarders, thickeners, plasticizers, superplasticizers, seeding materials and nanoparticle seeding materials.

In the context of the present disclosure derivative of a (chemical) component X is understood by any chemical modification which can be done with one single chemical reaction. One single chemical reaction is usually a one-step chemical process followed by purifica-tion. Single chemical reactions are for example, but not limiting, reduction oxidation, radi-cal substitution, electrophilic substitution, nucleophilic substitution, addition, elimination, isomerization, substitution, esterification, condensation, halogenation, hydrogenation, hy-drohalogenation, hydration, polymerization, dehydration.

Component (d) can also be a combination of any of the above-recited ingredients. Preferably, the additives in the mixture used according to the present disclosure (component (d)) as well as the biocarbon material (b) influence the reaction mechanism that leads to the hardening of the substrate. The interaction mechanism may be organic, inorganic or physical in nature and, for example, result in shrinkage reduction (shrinkage reducers), expansion (expansion agents), acceleration (accelerators), retardation (retarders), viscosity modification (e.g. plasticizers, thickeners), water retention (e.g. bleeding modifiers, wa-ter retention agents), water uptake (e.g. hydrophobizers), air entrainment (e.g. air enter-tainers, ventilators). The respective effect may be analyzed by means established in cement research (cf. e.g. Bauchemie, Plank et al, Chemische Technik-Prozesse und Produkte, Band 7: Industrieprodukte, Winnacker/Kiichler, 5. Auflage (2004) or associated DIN-Norms, e.g. DIN 1164-1).

Embodied herein is also a mixture comprising one or more biocarbon (b) and one or more additives (d) as a pre-mixture. Also embodied is a process of contacting biocarbon (b) and one or more additives (d) and subsequent contacting of this pre-mixture with one or more binders (a) and/or one or more aggregates (c). This pre-mixture is a preferred concrete ingredient replacer.

Preferred substances changing the reaction mechanism are a glyoxylic acid condensates and/or a glyoxylic acid adducts and/or glyoxylic acid, wherein sulfite addition products of glyoxylic acid are most preferred. Further preferred substances changing the reaction mechanism polyhydroxy compounds and/or salts or esters thereof, wherein the polyhydroxy compound is selected from polyalcohols with a carbon to oxygen ratio of C/O>1 and mixtures thereof.

Preferred retarders are saccharides, preferably monosaccahrides, preferably selected from the group comprising glucose, fructose, gluconate, disaccharides, preferably selected from the group comprising sucrose, lactose, maltose, trehalose, cellobiose, chito-biose trisaccahrides, preferably selected from the group comprising maltotriose, raffinose, meleziotose, and oligosaccharides.

Preferred plasticizers are lignosulfonates, maleinsulfonates, naphthalenesulfonates, sulfonated naphthalene formaldehyde condensates, sulfonated melamine formaldehyde condensate and acetone formaldehyde condensates.

Preferred superplasticizers are polycarboxylate ethers and polyarylic ethers.

Preferred seeding materials comprise microsilica and calcium silicate hydrate.

Preferred nanoparticle seeding materials comprise calcium silicate hydrate nanoparticles, preferably polymer stabilized calcium silicate hydrate nanoparticles.

Preferred accelerators are magnesium salt accelerators, preferably selected from the group comprising magnesium salts such as sulfates, nitrates, fluorides, chlorides and/or their hydrates, most preferably magnesium sulfate. Further preferred accelerators are al-kaline accelerators, alkali-free accelerators (AFAs) as well as chlorides, such as calcium chloride and magnesium chloride.

Table below shows an example of a flowable concrete mixture according to the invention.

TABLE 5
Parameters and compositon of a flowable concrete mixture

| | |
|---|---|
| Concrete type: | Flowable |
| Strength class: | C30/37 |
| Flow diameter: | 425 mm |
| Fresh density [kg/m3] | 2205 |
| w/c corr: | 0.66 |
| BWCP [%]: | 60 |
| Biocarbon content [kg/m3] | 80 |
| Biocarbon class | A |

| Volume: | Kg/m3 |
|---|---|
| Biocarbon | 80 |
| CEM I 42.5R | 350 |
| Fresh water | 229 |
| Aggregates 0-2 mm | 420 |
| Aggregates 2-8 mm | 534 |
| Aggregates 8-16 mm | 447 |
| Admixture PCE based | 3 |
| Total | 2063 |

According to a further aspect the invention relates to an optimization tool comprising program code means for performing a method according to the first aspect of the invention, when said tool is run on a computer. According to a further aspect the invention relates to a computer readable medium carrying a computer program comprising program code means for performing the method according to the first aspect of the invention, when said program product is run on a computer. The invention also relates to a control unit, which is able to perform the method according to the first aspect of the invention, especially to control the at least one step of processing of the at least one biocarbon and/or the concrete mixture.

In another aspect, the present disclosure provides a method of modifying an original concrete mixture, comprising the steps of: obtaining the original concrete mixture, desired properties of the concrete mixture, and desired performance parameters of the concrete mixture; analyzing a first database and a second database to determine a replacement biocarbon for at least one ingredient in the original concrete mixture, wherein the first database comprises physical and chemical properties of a plurality of biocarbons, and the second database comprises performance data for concrete compositions comprising a second plurality of biocarbons; outputting a set of parameters relating to the replacement biocarbon, wherein the set of parameters comprises material properties of the replacement biocarbon; and outputting a revised concrete mixture, wherein the revised concrete mixture comprises the replacement biocarbon. The analyzing step can comprise determining the replacement carbon by predicting the performance of the revised concrete mixture, based on the information in the first and second databases.

In another aspect, the present disclosure provides a digital optimization tool, comprising: a first database; a second database; and a processor. The first database comprises data relating to material characteristics of a first plurality of biocarbons. The second database comprises data relating to performance characteristics of concrete compositions comprises-ing a second plurality of biocarbons. The processor receives input(s) relating to an original concrete composition, analyzes the first and second databases to determine a replacement biocarbon for at least one ingredient in the original concrete composition, and outputs a revised concrete mixture. The revised concrete mixture has improved carbon emission and/or sequestration properties as compared to the original concrete composition. The processor can use machine learning and/or artificial intelligence to analyze the first and second databases and determine the replacement carbon.

### BRIEF DESCRIPTION OF THE FIGURES

FIG. 1 is a schematic drawing of a process according to the present disclosure.

FIG. 2 is a detailed schematic drawing of one of the steps in the process of FIG. 1.

FIGS. 3-12 show data that is used in the process of FIG. 1.

FIG. 13 is a detailed schematic drawing of a second one of the steps in the process of FIG. 1.

FIG. 14 shows an additional example of data that is used in the process of FIG. 1.

FIGS. 15a and 15b show examples of biocarbon particles with different aspect ratios.

FIG. 16 is a block diagram showing one possible structure of the digital optimization tool of the present disclosure.

FIGS. 17a-e show a process diagram for the method of FIG. 1, with detail shown in the process steps.

FIGS. 18a-18d show a process diagram for an alternative method of the present disclosure.

FIGS. 19-29 show different relationships used in the method according to the first aspect of the invention.

### DETAILED DESCRIPTION OF THE FIGURES

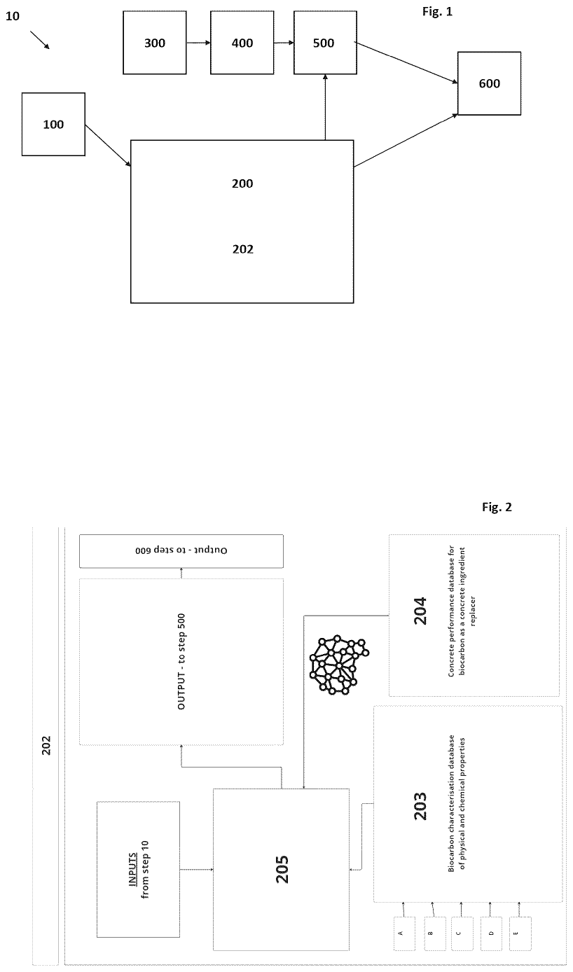

Referring to the drawings, and in particular FIG. 1, process 10 of the present disclosure is shown. In process 10, an original concrete mixture is adjusted through the use of data analysis to provide a revised concrete mixture that replaces commonly used inorganic materials (which emit significant amounts of carbon dioxide) with at least a certain percentage of biocarbon materials. Process 10 also determines desired characteristics of the biocarbon replacement materials, instructions on how to process them, and supplies those replacement materials. The present disclosure provides methods to characterize, treat and process biocarbon(s) and combinations thereof as a concrete ingredient replacer and concrete performance enhancer and predictor.

Accordingly, in step 100, an original concrete mixture, desired properties, and/or desired performance parameters for a new mix are obtained. In step 200, this information is fed into a digital optimization tool (DOT) 202. As discussed in greater detail below, DOT 202 takes this information and using an extensive database of properties of biocarbon materials, performance characteristics of concrete, outputs information relating to a replacement biocarbon to be used in a revised concrete mixture as a concrete ingredient replacer. As described in further detail below, DOT 202 can use algorithms or programs and data analysis and visualization tools to make this determination, and optionally, machine learning and artificial intelligence (AI). In step 500, the replacement biocarbon is prepared according to the parameters sent by DOT 202 in step 200. At this point, or at some point prior, biomass waste is processed into biocarbon, step 300, and in step 400, procured for the process of step 500. DOT 202 also outputs a revised concrete mixture in step 600. Steps 100 through 600 do not need to be taken in numerical order or the order in which they are described herein.

Referring to FIG. 2, a detailed schematic of DOT 202 is shown. DOT 202 has a first database 203 and a second database 204 therein. First database 203 includes a comprehensive range of biocarbon material characteristics, such as but not limited to particle size, surface area, porosity and chemical composition. These characteristics all play an essen-tial role in the prediction and selection of suitable biocarbon(s) for concrete ingredient replacement. Second database 204 includes prior usage data, including concrete mechanical performance and atmospheric carbon sequestration, which helps to quantify the biocarbon(s) concrete performance indicators. This prior usage data can include the performance of certain concrete compositions that include particular biocarbons. The prior usage data can be generated by the entity that owns and maintains DOT 202, or it can be imported from an external source.

The integration of databases 203 and 204 provides a way for DOT 202 to select and predict the effectiveness of using biocarbon(s) as a replacement for traditional concrete ingredients, offering significant potential for reducing the environmental impact of concrete production. Ultimately, the two databases facilitate the use of biocarbons as a concrete ingredient replacer for a variety of applications and provide the required supporting data for subsequent aspects of the disclosure.

Structuring data in the way described above, i.e. in separate databases 203 and 204, makes managing, accessing, and manipulating the data more manageable. However, the present disclosure contemplates that all of the data described herein can be incorporated into one database or data structure and organized according to content.

First database 203 includes material properties in the form of data on different biocarbon materials (biocarbon from different biomass feedstocks) as mentioned previously.

The construction of first database 203 includes cataloging the widest possible range of biocarbon material characteristics. In FIG. 2, this is shown as inputting characteristics about biocarbons A, B, C, D, E, etc. In one aspect of the present disclosure, DOT 202 can use one or more parameters from the aforementioned list, as well as information from database 203 if needed, as key performance indicators for the determination of a biocarbon material's potential as a concrete ingredient replacer. Each performance indicator has one or more associated predictive models, with each model based on prior usage and test data and comprising a quantifiable metric for the respective performance indicator.

Second database 204 includes data for biocarbon(s) performance as a concrete ingredient replacer and considers for example but not limited to the following concrete performance indicators:
Mechanical strength performance (7 and 28 day compressive and flexural strength)
Concrete rheological performance (workability/flowability)
Durability performance indicators (chloride resistance, depth of water pene-tration/permeability, freeze thaw resistance, acid attack, carbonation resistance, water absorption through capillarity)
Long-term stability indicators (pH fluctuations, internal carbonation resistance, long-term mechanical strength performance>28 days)
CO2 footprint performance
Performance with respect to different concrete ingredient material replacements and ratios of replacement
Performance with respect to the biocarbon(s) selection, the quantity and ra-tio(s) for maximum ingredient replacement and performance
Performance with different biocarbon blends and pre-treatments
Thermal conductivity performance
Acoustic conductivity performance
Leachate performance—the ability to reduce concrete contaminant leaching
Filtration performance—the ability of the concrete to filter pollutants/contami-nants from air/water
The density of concrete containing biocarbon(s)
The porosity of concrete containing biocarbon(s)
The setting time of cement pastes containing biocarbon(s)
The cement paste reactivity containing biocarbon(s) admixtures-calorimetry study

The biocarbon materials of the present disclosure are stored and analyzed in databases 203 and 204.

In the manner described in greater detail below, considering the above concrete performance indicators (from second database 204) as well as the properties of biocarbons, such as particle size, surface area, porosity, chemical composition, and moisture content et al. (from first database 203), allows for accurate prediction of concrete performance when in-corporating these materials. This prediction method permits the utilization of diverse biocarbon feedstock sources, whether independently or in conjunction, as concrete ingredient replacers across multiple applications. The integration of the databases 203 and 204 provides a means of selecting and predicting the suitability of biocarbon(s) as concrete ingredient replacers.

Based on the integration of the databases, a biocarbon or blend thereof is assessed for close characteristic similarities and based on past performance data. DOT 202 is able to provide a list of the biocarbon and concrete performance indicators as well as mix design optimizations according to the performance indicators considered and prioritized by a user (the latter as input during step 100). Databases 203 and 204 are constructed for machine readability and for use with programs and data analysis and visualization tools 205. In one aspect of the present disclosure, tools 205 can further include artificial intelligence (AI) and machine learning (ML). Tools 205 within DOT 202 are used for intelligent pattern recognition, data analysis and concrete mixture design and performance predictions using biocarbon as a concrete ingredient replacer. Tools 205 will be used to enhance the intelligence of DOT 202. For example with AI/ML, tools 205 can cross-reference biocarbon ash contents with surface area with workability, and possibly thermal conductivity as a means to predict strength performance. The many variables that go into selecting a suitable biocarbon for a particular application, in specified amounts and with specified characteristics, make that selection exceptionally complex and time-consuming to do without the use of tools 205. In database 203, there can be as many as several dozen material characteristics per biocarbon type. Tools 205 can use complex pattern recognition to increase the predictability by performing analyses across 203 and 204. DOT 202 will grow smarter with each new input datapoint and test result increasing the accuracy of mix design performance prediction.

In one aspect, the present disclosure provides a method to intelligently select and define biocarbon(s) by their chemical and or morphological characterization for utilization as a concrete ingredient replacer and by predicting and controlling for the physical performance properties of concrete using that concrete ingredient replacer. This method involves iden-tifying key performance indicators for biocarbon(s) based on its/their chemical and/or morphological properties and for concrete based on a set of testing protocols. These indicators enable the prediction and control of the physical and performance properties of the resulting concrete. The performance indicators are identified by integrating the two databases previously discussed, namely a biocarbon materials characteristic database (203) and a biocarbon concrete performance database (204). The construction and operation of these databases (203, 204) as well as the methods required to predict the performance of a biocarbon and its subsequent suitability as a concrete ingredient replacer are embodied herein.

“Cement/binder replacer” in the context of the present disclosure relates to the ability of a biocarbon material or materials to replace cement or other concrete binder, within concrete formulations without negatively affecting the concrete properties. Each biocarbon material or materials is assessed based on their chemical and physical characteristics and within their predicted performance for use in this context. Biocarbon material treatment processes for optimized and enhanced performance as a cement replacer are also considered. One aspect of the present disclosure is improving the concrete properties by replacing cement within the concrete with specific biocarbons. The selection and/or of the specific biocarbons combinations is described throughout.

“Aggregate replacer” in the context of the present disclosure relates to the ability of a biocarbon material or materials to replace aggregates within concrete formulations without negatively affecting the concrete properties. Aggregates comprise any sized aggregate material, e.g. but not limited to, silicate-based aggregates, granite-derived aggregates and gravels, recycled aggregates including recycled concrete, and lightweight aggregates. Each biocarbon material or materials are assessed based on their chemical and physical characteristics and within their predicted performance for use in this context. Biocarbon material treatment processes for optimized and enhanced performance as an aggregate replacer are also considered. One aspect of the present disclosure is improving the concrete and/or mortar properties by replacing aggregates within the concrete with specific biocarbons. The selection of the specific biocarbons and or combinations is described throughout.

A “concrete ingredient replacer”, “replacement concrete ingredient”, or “materials ingredient replacer” refers to a cement/binder replacer, an aggregate replacer, or a replacement for any of the ingredients in an original concrete mixture.

The suitability of each biocarbon material is assessed against multiple performance indicators drawing on data from the specific performance indictor databases described in detail below. It is embodied, that the property of biocarbon being a good concrete ingredient replacer is predicted based on performance indicators as inputs to the machine learning processing, such as those performance indicators described above with respect to database 204.

DOT 202 will perform these analyses based on the information in databases 203 and 204, and using tools 205. Based on these analyses, it will output an optimized biocarbon revised concrete mixture, as in step 600. It will also output information relating to the processing of the biocarbon, as in step 500. This processing information can include, but is not limited to, any of the information discussed above with respect to databases 202 and 203 or the following:
Cement type and or suitability
Additives type and dose and or suitability
Water/Cement ratio and or water correction factor
Quantity & blend of biocarbon materials
Particle sizes of biocarbon materials
Cement percentage replacement
Aggregate(s) percentage replacement
CO2 footprint of the final mix
Concrete performance prediction
Key biocarbon performance indicators, e.g. ash content, specific surface area, carbon content, and silica content

As implied in the above list, DOT 202 will not always output that the standard, inorganic cement or aggregate ingredients of a particular concrete mixture should be entirely replaced. In one aspect, the replacement of the cement fraction of the original concrete mixture with a biocarbon is between 1% to 30%, and/or the replacement of the aggregate fractions is between 1% and 20%, or any subranges therebetween.

Another aspect of the present disclosure is a method to establish concrete performance indicators of the revised concrete mixture. This method includes a description of databases 203 and 204, and identification of the performance indicators with a quantifiable metric determined and based on associated material testing protocols. The databases 203 and 204 are constructed for machine readability and for use with tools 205, namely programs and data analysis and visualization tools, machine learning algorithms for intelligent pattern recognition, data analysis and concrete mixture design and performance predictions using biocarbon as a concrete ingredient replacer.

Mechanical strength performance is one of the concrete performance indicators provided by the present disclosure. Biocarbon(s) are integrated into a standard mixture design and assessed according to common norm testing protocols (e.g. EN 196, EN 12350, EN 12390). The testing protocol considers the following assessments with the resulting data feeding into 204 to support 203 biocarbon assessment and for the outputs calculated by tools 205. Strength refers to both compressive and flexural strength assessments.
a. 7&28 day strength for biocarbon(s) as a cement replacer
b. 7&28 day strength for biocarbon(s) as a aggregates replacer
c. 7&28 day strength for biocarbon blends (different combinations and ratios) as a concrete ingredient replacer also considering both cement and aggregate replacements and associated ratio differences
d. 7&28 day strength for different biocarbon(s) pre-treated to contain varying moisture contents
e. 7&28 day strength for different biocarbon(s) pre-processed for different particle size distributions
f. 7&28 day strength effects due to the replacement of different EN 197-1 defined cement types with biocarbon(s).
g. 7&28 day strength for the use of biocarbon(s) in combination with other concrete ingredients such as chemical and mineral-based additive ingredients including superplasticizers, viscosity modifying agents, fly ash, blast furnace slag, calcined clays.
h. 7&28 day strength for the use of biocarbon(s) with additional CO2 reduction technologies such as CO2 concrete curing, CO2 mineralization/biomineralization, recycled aggregates, biogenic limestone

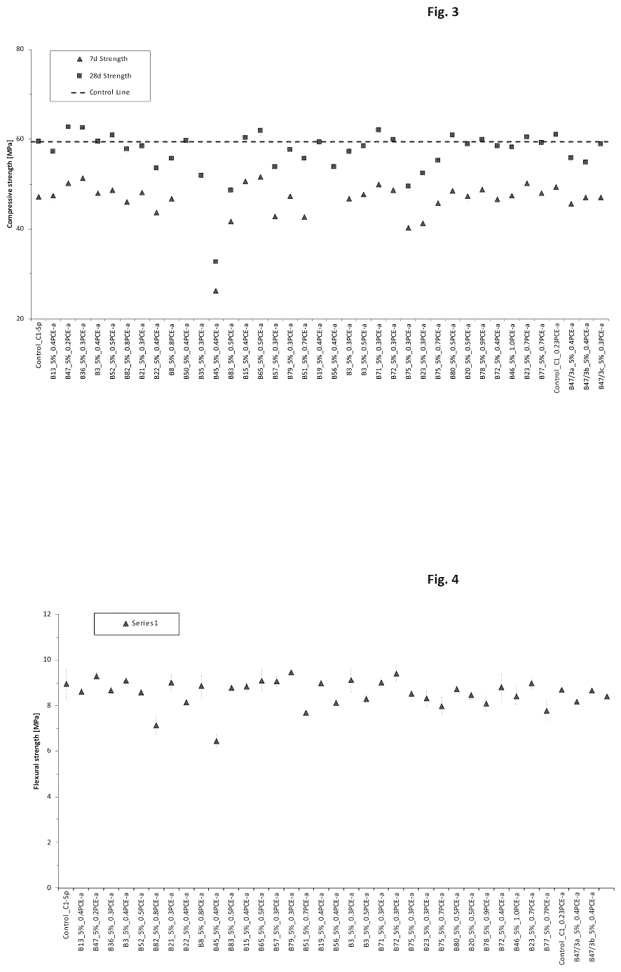

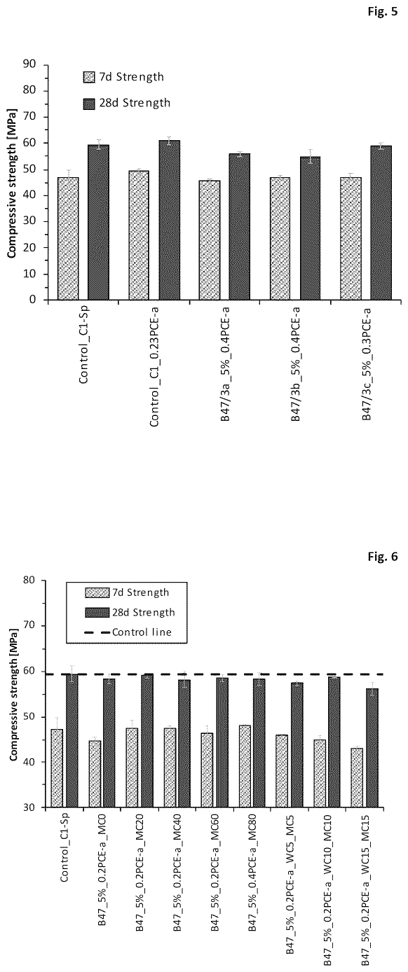

Example data used to build database 204 is shown in FIGS. 3-6. FIG. 3 shows 7&28 day compressive strength for certain biocarbon(s) used as a cement replacer. FIG. 4 shows 7-day flexural strength for biocarbon(s) used as a cement replacer. FIG. 5 shows 7&28 day compressive strength for different biocarbon blends used as a cement replacer. FIG. 6 shows 7&28 day compressive strength for different biocarbon water pre-treatments and then used as a cement replacer. The samples of FIGS. 3-6 were prepared and tested according to EN 196-1.

The rheological performance of fresh concrete is also a concrete performance indicator provided by the present disclosure. Biocarbon(s) are integrated into a standard mixture design and assessed according to common norm testing protocols (e.g. EN 1015, EN 12350). The testing protocol considers the following assessments with results from these supporting 204. Rheological performance includes but is not limited to the following:
Workability/flowability (flow table tests)
Presence of segregation (visual observations and image analysis)
Presence of bleeding (visual observations and image analysis)

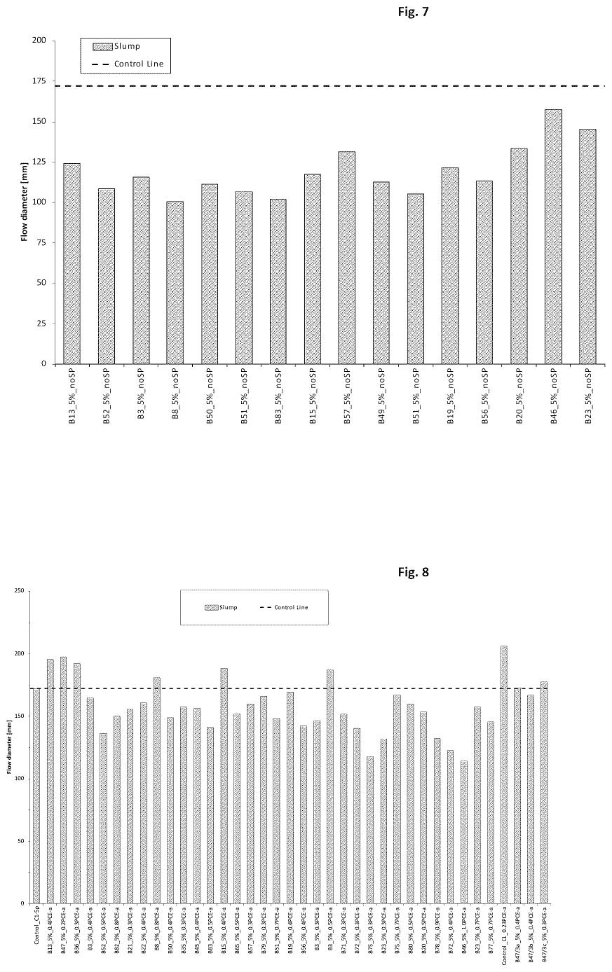

FIG. 7 shows fresh concrete flowability measurements for different biocarbon(s), without the use of superplasticizer and FIG. 8 shows fresh concrete flowability measurements for different biocarbon(s), with the use of superplasticizer. The samples of FIGS. 7 and 8 were prepared and tested according to EN 1015.

Long-term stability measures such as pH fluctuations, long-term structural strength assessments and freeze thaw performance are also concrete performance indicators in the present disclosure.

An example of pH fluctuation data used to support database 204 is provided below. Bio-carbon(s) are integrated into a pre-defined high biocarbon mixture design and assessed at pre-defined time intervals such as after 3, 6, 12, 18, 24, 36, 48 months.

After each time interval, the samples are split open and a solution containing phenolphthalein is applied to the freshly exposed surface. The phenolphthalein turns the surface pink to fuchsia for pH levels between 8.3-10. Outside of this range no color change occurs. Special attention is paid to the area of material surrounding the biocarbon particles and in case of carbonation an area of colorless material would occur. The carbonation boundaries are assessed using high magnification imagery with a boundary measurement.

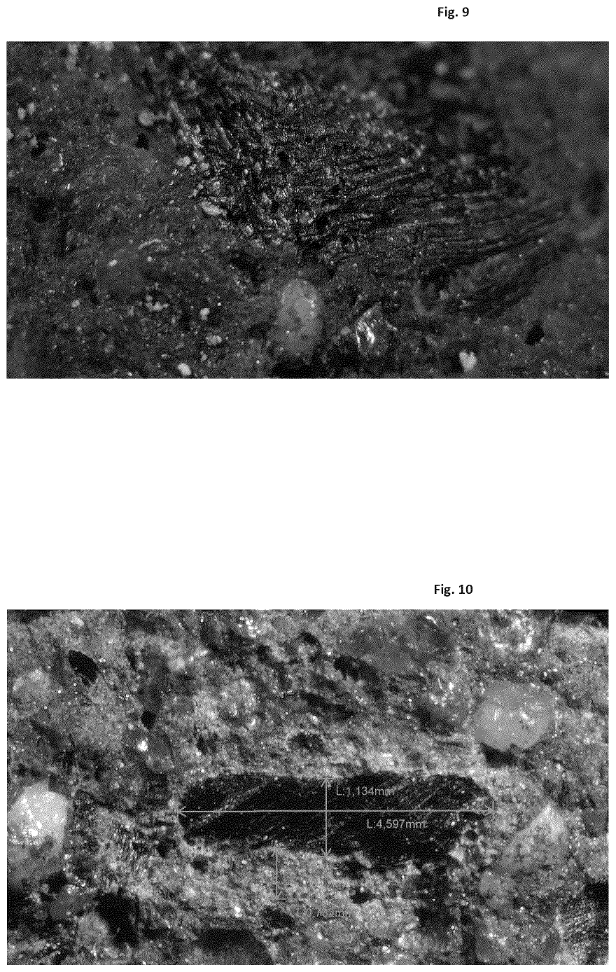

FIG. 9 shows an example of an observation in a concrete sample where no carbonation has occurred after 3 months. This is indicated by the absence of a grey zone between the biocarbon particle and the adjoining cement matrix. FIG. 10 shows an example where carbonation has occurred after 3 months. This is indicated by the presence of a grey zone around the biocarbon particle. The extent of carbonation is assessed according to the dis-tance between the biocarbon particle to the limits of the grey zone measured in the normal direction from the biochar particle.

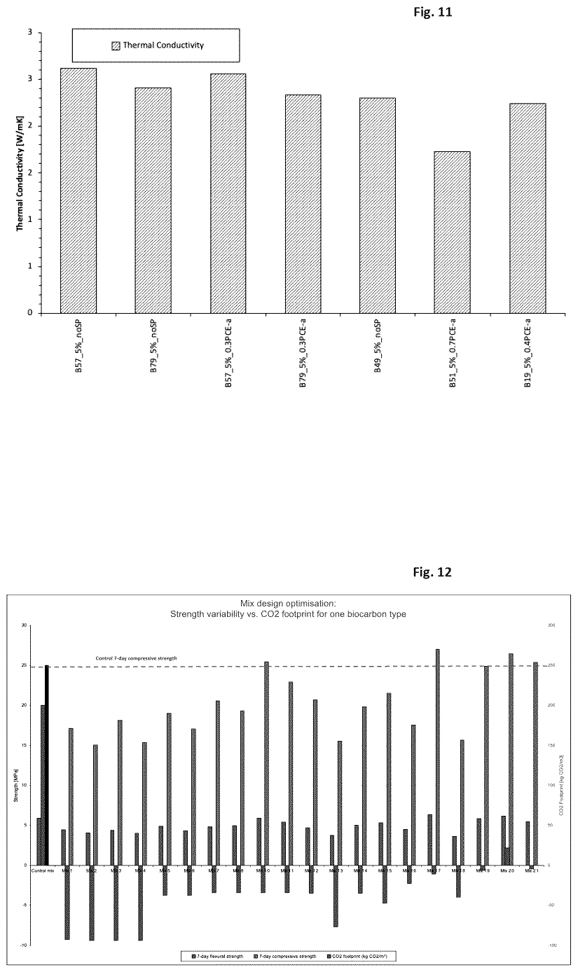

The thermal conductivity performance of concrete where biocarbon has been used as a materials ingredient replacer is also a concrete performance indicator of the present disclosure. The present disclosure has discovered that a correlation exists between the biocarbon characterization (including for blended biocarbons), biocarbon mix quantities, pre-treatment processes and thermal conductivity of biocarbon amended concrete. Biocar-bon(s) are integrated into a pre-defined biocarbon(s) amended mix design and assessed after 28 days for their thermal conductivity in W/mK. FIG. 11 shows thermal conductivity values for a standard concrete mix and different biocarbons.

The CO2 footprint of the concrete ingredient materials and final cubic meter CO2 footprint is also a concrete performance indicator of the present disclosure. Performance of the biocarbon(s) as amended concrete materials is assessed according to global warming potential values for all mix ingredients in kg CO2/kg or kg CO2/m³. This information is integrated into database 204 and used to assess a biocarbon amended mix design for its final CO2 footprint performance as well as a biocarbon or biocarbon blend for its CO2 reduction potential. FIG. 12 shows example CO2 footprint data.

Other potential concrete performance indicators according to the present disclosure include, but are not limited to those referenced as part of the 204 description above.

Methods according to the present disclosure that are used to determine the biocarbon performance indicators is described below. For each of the concrete performance indicators, the biocarbon or biocarbon blends used as a concrete ingredient replacer is/are assessed against its/their chemical and/or morphological properties. Depending on the concrete performance results, the key biocarbon performance indicators are identified.

In one aspect of the present disclosure, to determine the mechanical strength performance indicator(s) of a particular biocarbon(s), the following factors are considered by DOT 202 (list not exhaustive):
a. Feedstock material
b. Average feedstock processing temperature
C. Carbon content
d. Ash content
e. Oxide content
f. Surface morphology and surface charge
g. Moisture content (%) of the biocarbon (partial or water full saturation)
h. pH (at integration and variability over time)
i. Water holding capacity

In one aspect of the present disclosure, to determine the rheological performance indica-tor(s) of a particular biocarbon(s), the following factors are considered by DOT 202 (list not exhaustive):
a. Moisture content (%) of the biocarbon (partial or water full saturation)
b. Water holding capacity
C. Particle size distribution
d. Particle size and shape
e. Specific surface area
f. Ash content
g. Carbon content
h. pH (at integration and fluctuations over time)
i. Zeta potential

To determine the long-term stability performance indicator(s) of a particular biocarbon(s), the following factors are considered by DOT 202 (list not exhaustive):
a. Feedstock material
b. Average feedstock processing temperature
C. Carbon content
d. Ash content
e. Ash composition
f. Oxide content
g. Oxygen/carbon ratio
h. Hydrogen/carbon ratio
i. Surface morphology and surface charge
j. Zeta potential
k. pH (at integration and fluctuations over time)

The present disclosure has discovered that a correlation exists between the biocarbon characterization (including for blended biocarbons), biocarbon mix quantities, pre-treatment processes and thermal conductivity of biocarbon amended concrete.

To determine the thermal conductivity performance indicator(s) of a particular biocarbon(s), the following factors are considered by DOT 202 (list not exhaustive):
Specific density
Specific surface area
Carbon content
Ash content

To determine the CO2 performance indicator(s) of a particular biocarbon(s), the following factors are considered by DOT 202 (list not exhaustive):
a. Carbon Content
b. Silica content
c. Ash content

The aspects of the immediately preceding paragraphs are examples of how DOT 202 will determine concrete ingredient replacers. The present disclosure contemplates the use of other parameters for those calculations or a different order of influence.

The present disclosure can also provide concrete thermal conductivity performance predictability based on the biocarbon thermal conductivity predictive indicators. The present disclosure can also enhance the thermal insulation properties of concrete though predictive modelling for enhanced concrete performance.

The present disclosure also provides a method of prediction and control of the atmospheric carbon sequestration properties of concrete utilizing one or more biocarbon(s) as a concrete ingredient replacer characterized by chemical and or morphological properties.

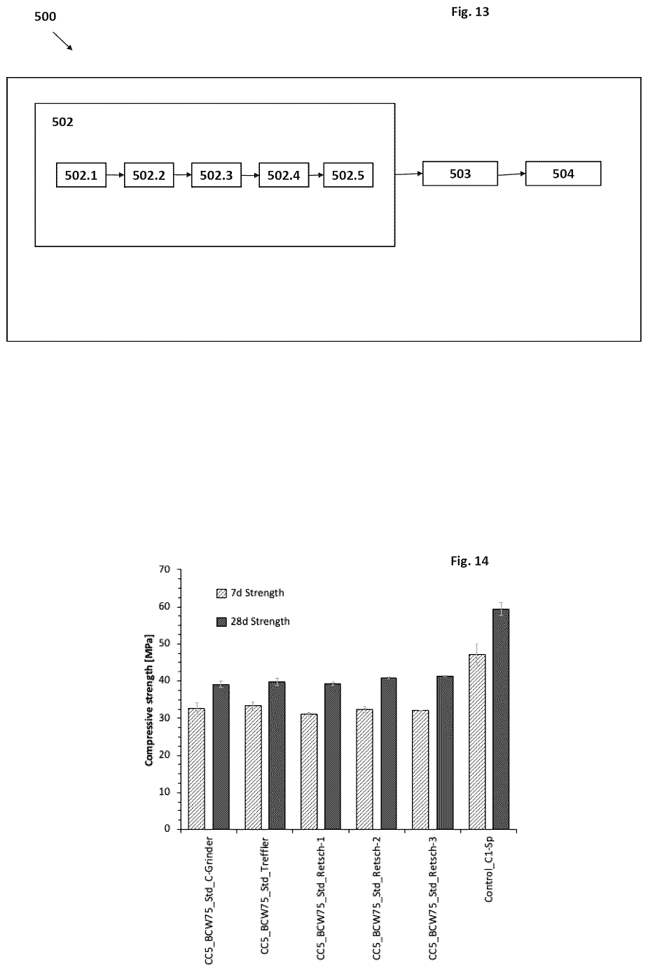

Referring now to FIG. 13, a detailed schematic of step 500 is shown. As noted above, step 500 of process 10 is directed to the pretreatment of the replacement biocarbon, according to the output provided by DOT 202. In sub-step 502, the selected biocarbon(s) are processed for optimum performance in the new concrete. This processing contains individual treatments 502.1, 502.2, 502.3, 502.4, and 502.5, each described in greater detail below.

Treatment 502.1 is a method for pre-treating biocarbon(s) with water to optimize both fresh and hardened performance characteristics of concrete by the replacement of interstitial gas volumes in and around the biocarbon(s) thereby modifying the quantities of additives required. It is another aspect of the present disclosure that a correlation exists between the biocarbon material characteristics and the level of moisture that can be held within the biocarbon material during the mixing process. Consequently, the properties to influence the final concrete and/or mortar properties of the different biocarbon materials can be correlated to their interaction with water, preferably with their water holding capacity. In treatment 502.1, the biocarbon(s) are combined with a very specific percentage of pre-added moisture. The percentage moisture content added is based on the ratios of the different types of biocarbon and their resulting combined water retention capacity.

The present disclosure also contemplates a further correction factor, namely biocarbon water absorption factor (BWAF), where partial or full saturation of the biocarbon(s) with the additional water is not practical or possible. In this situation the additional water can be added to the concrete mix directly. Here the quantity of water to be “held” by the biocarbon materials will be reduced depending on the following but not limited to:
Biocarbon saturation rate
Water holding capacity.
Specific surface area
Zeta potential
Hydrophobic or hydrophilic properties

The present disclosure has discovered that a correlation exists between the biocarbon material characteristics and the level of moisture that can be held within the biocarbon material during the mixing process when partial to full pre-saturation is not undertaken. When the biocarbon material has been partially or fully pre-saturated the BWAF=1.

While the BWCF and BWAF are concerned with additional water added on top of the mixing water the partial or complete saturation technology is concerned with the biocarbon materials integration regardless of whether or not a BWCF and BWAF are applied.

Without being bound by theory, it is believed that partial or complete biocarbon water pre-saturation provides the additional benefits:

Increase in concrete structural strength performance
Increased workability without compromising 7/28-day strength performance
Reduces the required dose of superplasticizer
To provide mix moisture for a more efficient binder use for later stage hydration reactions and for increased later stage strength developments, i.e. provides internal curing

Where full pre-saturation is not possible, the level of partial pre-saturation for optimized integration and performance is dependent and is correlated to the following biocarbon material characteristics. When blended biocarbon materials are used, it is the combined characteristics that are taken into account.
Water holding capacity (WHC)
pecific surface area (SSA)
Ash Content

In an aspect of treatment 502.1, the biocarbon material is saturated or partially saturated with the additional moisture in advance of concrete addition. The additional water is applied to the biocarbon in advance of its use and with sufficient time to reach partial saturation or saturation equilibrium. Mechanical mixing or temperature controlling can be used to accelerate the process. This is to provide a homogeneous distribution and absorption throughout the micro and macro pore structures of the biocarbon material.

One alternative treatment 502.1 includes a method for pre-treating biocarbon with water or steam to optimize both fresh and hardened performance characteristics of concrete by en-hancing the surface properties of biocarbon. A third treatment 502.1 is a method for pre-treating biocarbon with gas purges to control biochar reactivity and enhance concrete properties and performance.

The present disclosure has discovered that a correlation exists between the biocarbon's internal gas composition and the following concrete performance indicators (list not exhaustive):
Mechanical strength (compressive and flexural)
Durability (chloride ingress, depth of water penetration/permeability, freeze thaw, acid attack, carbonation, water absorption through capillarity)

In this treatment, the biocarbon(s) are purged prior to integration with the following gasses: N2, CO2, Steam, and any combinations thereof. The present disclosure may also provide performance predictability based on the applied gas saturation pre-treatment for enhanced concrete performance.

In treatment 502.2, the present disclosure provides a method for pre-processing biocar-bon(s) for mechanical design optimizations by particle size reduction and controlling, as well as particle shape design and controlling for the use of biocarbon(s) as a concrete ingredient replacer and predictive performance estimation(s). The present disclosure has discovered that there is a correlation between the biocarbon's particle size distribution (PSD) measured according to ISO 13320:2020 or via a sieving analysis (EN 933-1 or ISO 2591-1:1988 or ISO 3310-1:2016 and optional ISO 9276-2:2014 for visualization) and the following concrete performance indicators (list not exhaustive):
Mechanical strength (compressive and flexural)
Workability and flowability
Durability (chloride ingress, depth of water penetration/permeability, freeze thaw, acid attack, carbonation, water absorption through capillarity)·
Thermal conductivity

Thus, DOT 202 can use such correlations to optimize the biocarbon(s) particle size distribution (PSD) to enhance concrete performance characteristics including but not limited to the list above.

The present disclosure may also provide concrete performance predictability based on the PSD profile and in addition to enable material optimization for enhanced concrete performance. FIG. 14 shows an example of the performance of one biocarbon material type with a different PSD and assessed according to the concrete strength performance indicator(s) (compressive and flexural strengths). This data is used to provide performance predictability based on the biocarbon type blends and ratios also for biocarbon type blends with additives for enhanced concrete performance, another aspect of this disclosure.

The present disclosure has also discovered that a correlation exists between the biocarbon's particle shape factors measured according to ISO 19749:2021, including but not limited to aspect ratio (AR), and the following concrete performance indicators (list not exhaustive):
Mechanical strength (compressive and flexural)
Workability and flowability
Durability (chloride ingress, depth of water penetration/permeability, freeze thaw, acid attack, carbonation, water absorption through capillarity)

Irregularly shaped and angular particles can compromise workability while improving packing density. Spherical or rounded particles enhance workability. In addition, particle shape can affect the surface area and porosity of the biocarbon particles, which can influence the concrete's water demand, setting time, and overall performance.

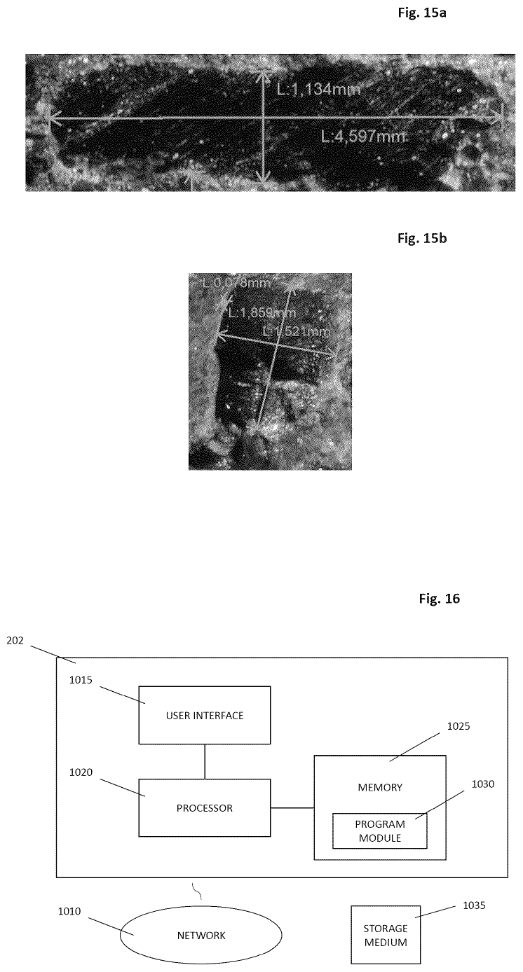

FIG. 15a shows a biocarbon particle with an AR of 4.0, and FIG. 15b shows one with an AR of 1.2. Another aspect of the present disclosure is to provide performance predictability based on the particle shape profiles and in addition to enable material optimization for enhanced concrete performance.

In treatment 502.3, different biocarbons can be mixed if applicable. Treatment 502.4 is directed to preparation of the biocarbons for packaging, shipping, and storage, and mitigation of any risks such as flammability and explosiveness. Treatment 502.5 comprises a compliance check with quality assurance standards. Not all of treatments 502.1 through 502.5 are mandatory, and one or more can be performed in sub-step 502. In sub-steps 503 and 504, the biocarbons are shipped and delivered, respectively.

Referring back to FIG. 1, in step 300, biomass waste is collected from several possible sources, as mentioned previously. In step 400, the biomass waste is converted thermally to biocarbon. The thermal conversion processes can be any of those described in Table 1 or any combinations thereof. Step 400 can occur at the same facility as the treatments of sub-step 500, or a different one.

In the present disclosure, an example of a revised concrete mixture comprises the following ingredients or consists of the following components:
(a) cementitious based binder, preferably silicate cement, Portland cement, CEM I, CEM II, CEM III, CEM IV, CEM V, CEM VI and combinations thereof;
(b) one or more biocarbon materials;
(c) one or more aggregates, preferably selected from the group comprising, gravel, silt, sand, fines, light weight aggregates;
(d) optionally, one or more additives; and
(e) optionally, water.

An example of an original concrete mixture comprises the following ingredients or consists of the following ingredients:
(a1) cementitious based binder, preferably silicate cement, Portland cement, CEM I, CEM II, CEM III, CEM IV, CEM V, CEM VI and combinations thereof;
(b1) one or more aggregates, preferably selected from the group comprising, gravel, silt, sand, fines, light weight aggregates;
(c1) optionally, one or more additives; and
(d1) optionally, water.

One example of the present disclosure concerns a 60 MPa mix for pre-cast products as earth moist concrete ingredient replacer. This example uses a blend of biocarbons as a cement and or aggregate replacer to produce pre-cast earth moist concrete with the biocarbon material comprising of the following. The biocarbon blended mix consists of 50% of a biocarbon material with an ash content>50% and 50% of a biocarbon material with an ash content≤50%. The biocarbon blended mix has a moisture content of between 10% and 55%. The biocarbon blended mix has a particle size≤300 and with a D50 between 0.2 mm and 1 mm.

The biocarbon blended mix replaces more than 2% and less than 10% of cement and more than 2% and less than 20% fine aggregates (0-2 mm grain size) in the moist earth mix. An aspect of the present disclosure is for the biocarbon blended mix to replace 5% of cement and 10% fine aggregates (0-2 mm grain size) in the moist earth mix. Another aspect of the present disclosure is for the biocarbon blended mix to replace 5% of cement and between 5% and 10% of the total aggregate ingredients covering all grain sizes and types in the moist earth mix with the percentage of each aggregates reduced proportionally.

Table below shows an example CO2 neutral mix design for a C25/30 strength grade.

TABLE 6
CO2 neutral mix design for a C25/30 strength grade

| | |
|---|---|
| Project name: | Project example |
| Application: | Application example |
| Strength class: | C25/30 |
| Flow class: | Flow class example |
| Air content: | 2% |
| w/c eq: | 0.64 |
| CO2 footprint | −1.48 |
| [kg CO2/kg] | |
| CO2 footprint | 101% |
| reduction [%] | |

| | Volume: | | |
|---|---|---|---|
| | 1 m³ (dry) | 1 m³ (inc. moisture) | |
| Raw materials: | kg/m³ | M.-% | kg |
| eL Materials | 60.83 | 20% | 76.03 |
| CEM III/A 42.5N Slag cement | 258.20 | | 258.20 |
| Limestone Powder | 49.60 | | 49.60 |
| Fresh water | 164.99 | | 149.78 |
| Aggregates 0-2 mm | 694.31 | 0.00% | 694.31 |
| Aggregates 2-8 mm | 346.67 | 0.00% | 346.67 |
| Aggregates 8-16 mm | 694.31 | 0.00% | 694.31 |
| Admixture A | 2.58 | | 2.58 |
| Admixture B | 1.29 | | 1.29 |
| Total | 2272.78 | | 2272.78 |

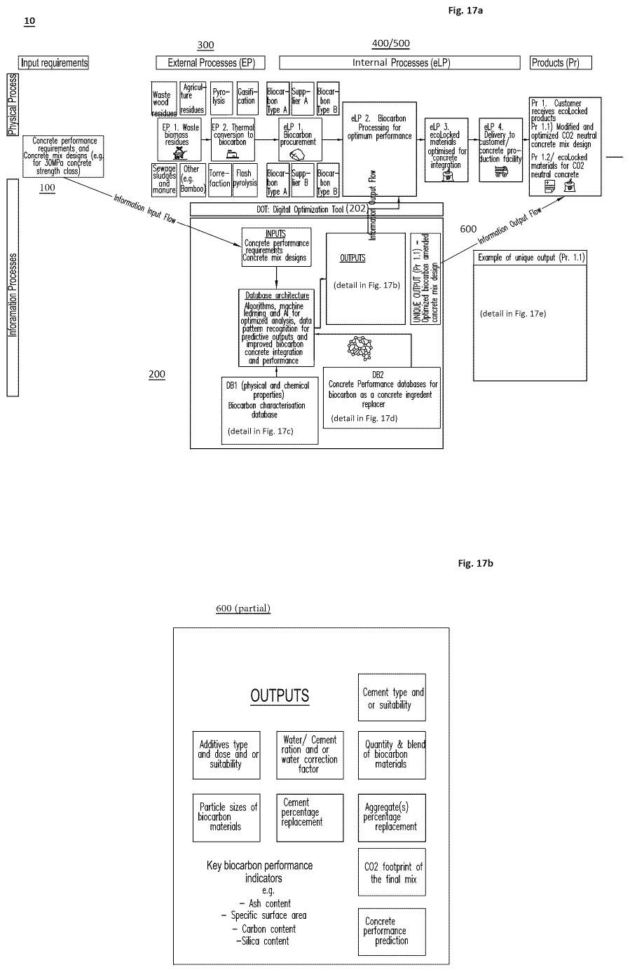

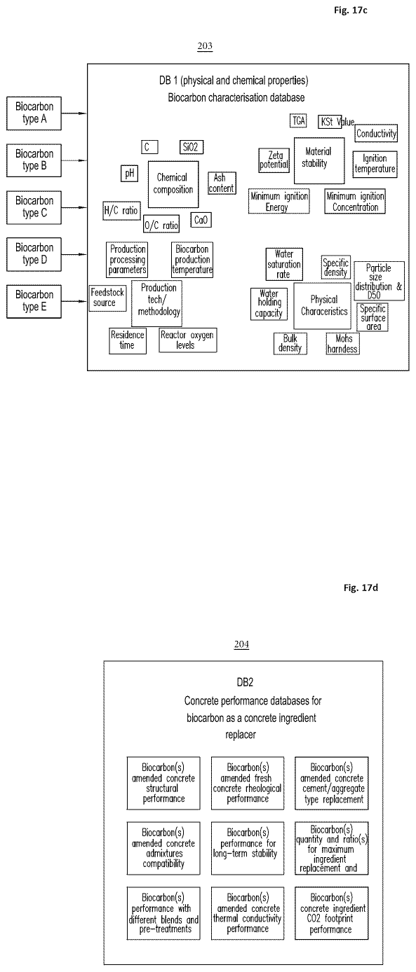

FIG. 16 is an additional block diagram of DOT 202. DOT 202 is coupled to a network 1010, e.g., the Internet.

DOT 202 can have a user interface 1015, a processor 1020, and a memory 1025. DOT 202 may be implemented or reside on a general-purpose microcomputer. DOT 202 can be coupled to other devices (not shown) via network 1010. Processor 1020 is configured of logic circuitry that responds to and executes instructions, for example tools 205 discussed earlier.

Memory 1025 stores data and instructions for controlling the operation of processor 1020. Memory 1025 may be implemented in a random access memory (RAM), a hard drive, a read only memory (ROM), or a combination thereof. One of the components of memory 1025 is a program module 1030.

Program module 1030 contains instructions for controlling processor 1020 to execute the methods described herein. For example, as a result of execution of program module 1030, processor 1020 uses tools 205, and the information described above from first database 203 and second database 204 to determine the appropriate biocarbon material for a particular application. The term “module” is used herein to denote a functional operation that may be embodied either as a stand-alone component or as an integrated configuration of a plurality of sub-ordinate components. Thus, program module 1030 may be implemented as a single module or as a plurality of modules that operate in cooperation with one another. Moreover, although program module 1030 is described herein as being installed in memory 1025, and therefore being implemented in software, it could be implemented in any of hard-ware (e.g., electronic circuitry), firmware, software, or a combination thereof.

User interface 1015 includes an input device, such as a keyboard or speech recognition subsystem, for enabling a user to communicate information and command selections to processor 1020. User interface 1015 also includes an output device such as a display or a printer. A cursor control such as a mouse, track-ball, or joy stick, allows the user to manipulate a cursor on the display for communicating additional information and command selections to processor 1015.

Processor 1020 outputs, to user interface 1015, a result of an execution of the methods described herein. Alternatively, processor 1020 could direct the output to a remote device (not shown) via network 1010, as shown and previously discussed in step 600.

While program module 1030 is indicated as already loaded into memory 1025, it may be configured on a storage medium 1035 for subsequent loading into memory 1025. Storage medium 1035 can be any conventional storage medium that stores program module 1030 thereon in tangible form. Examples of storage medium 1035 include a floppy disk, a compact disk, a magnetic tape, a read only memory, an optical storage media, universal serial bus (USB) flash drive, a digital versatile disc, or a zip drive. Alternatively, storage medium 1035 can be a random access memory, or other type of electronic storage, located on a remote storage system and coupled to DOT 202 via network 1010.

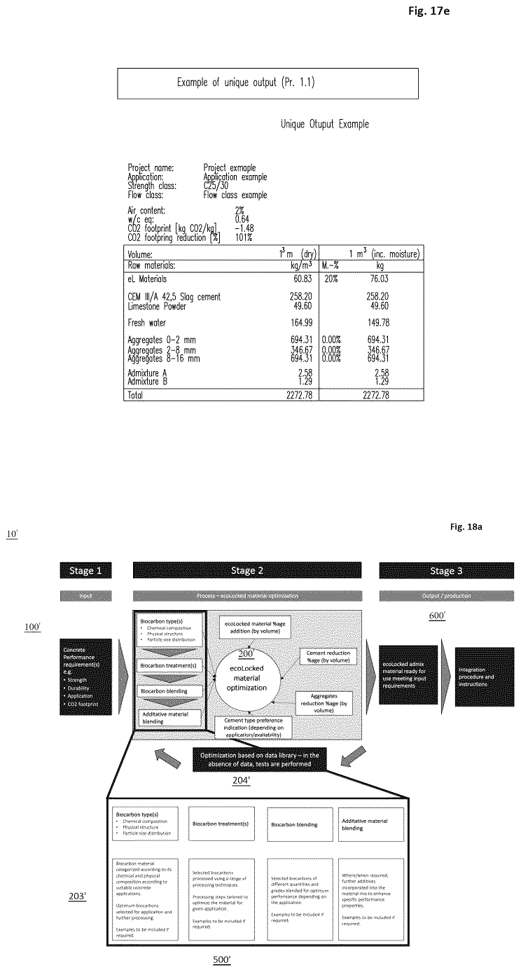

Referring to FIGS. 17a-17e, a detailed diagram of process 10 is shown. FIG. 17a depicts steps 100, 200, 300, 400, 500, and 600, as well as DOT 202. FIG. 17b shows an example of the output provided in step 600, in detail. FIG. 17c shows a detailed conception of what is stored in first database 203. FIG. 17d shows a detailed conception of what is stored in second database 204. FIG. 17e shows an example of a revised concrete mixture that is output in step 600.

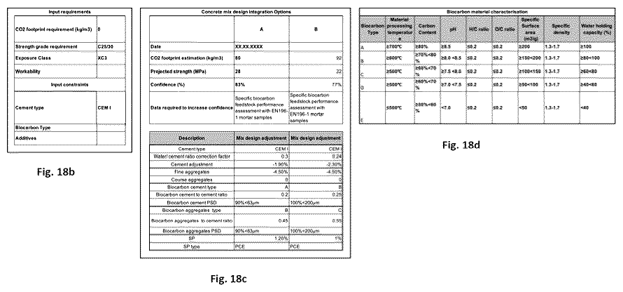

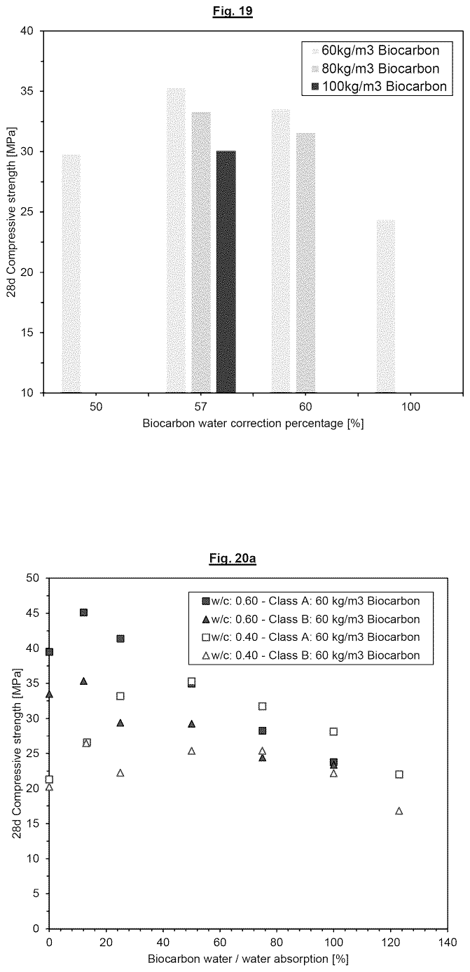

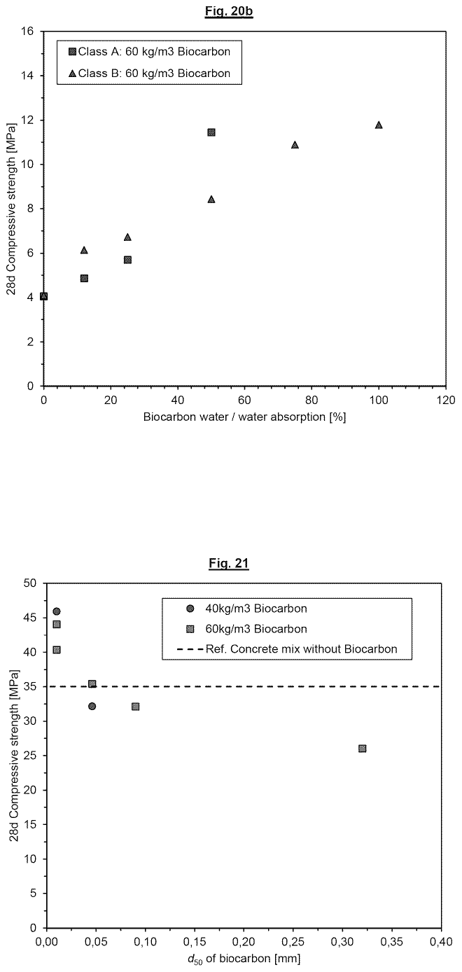

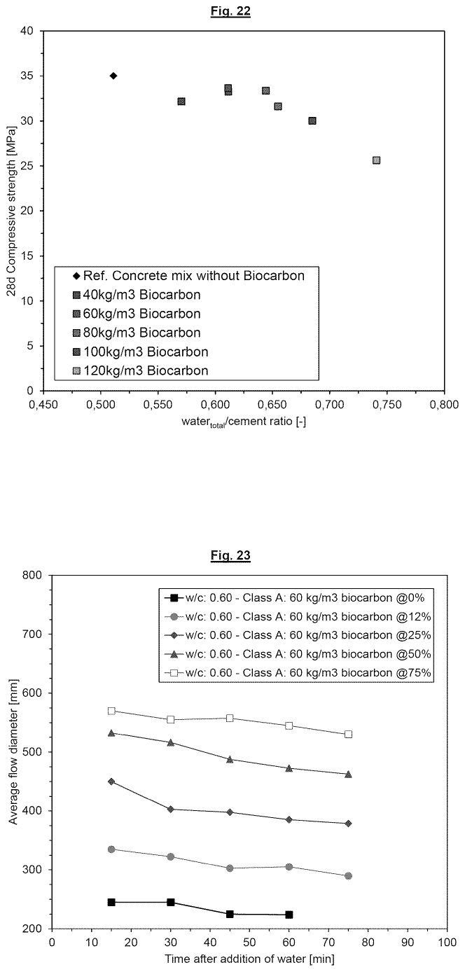

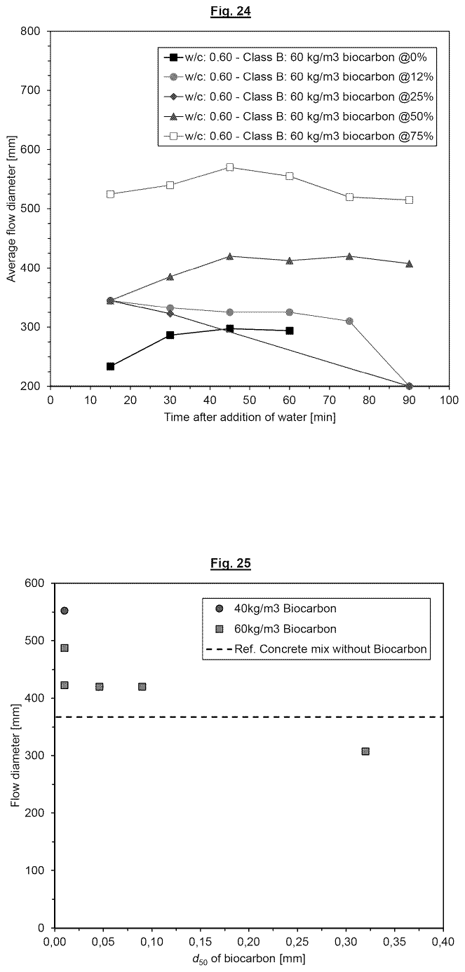

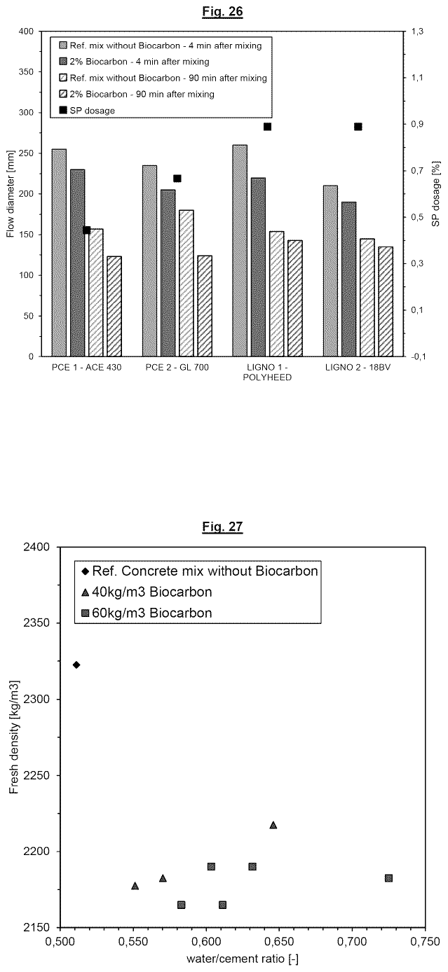

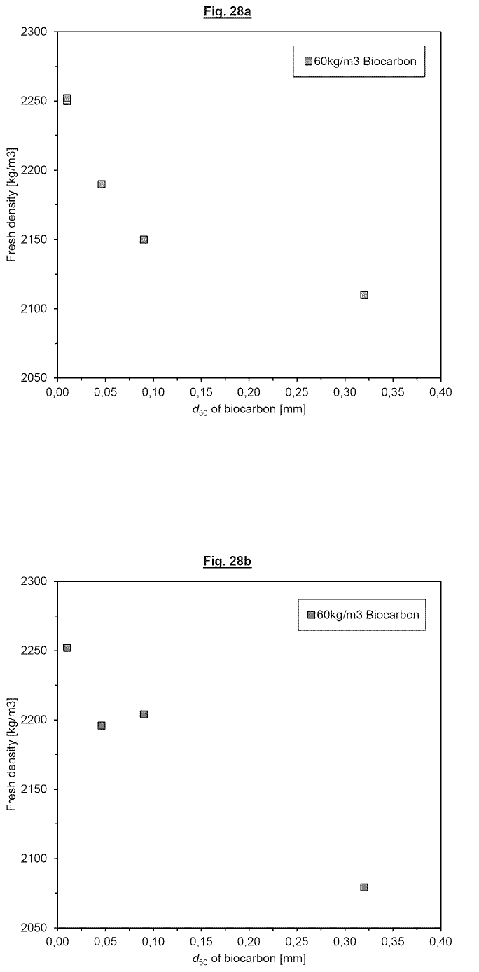

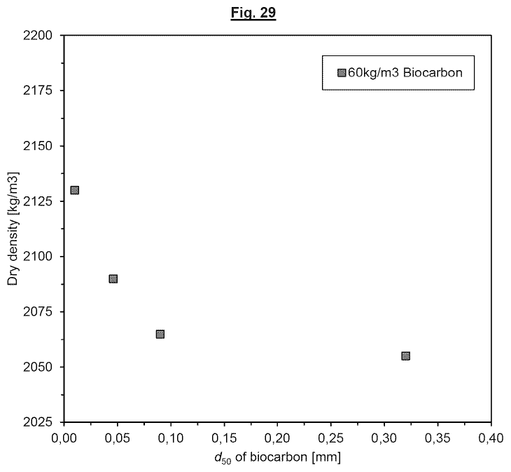

Referring to FIGS. 18a-18d, a schematic diagram of a process 10′ according to the present disclosure is shown. Process 10′ is similar to process 10, and has similar input step 100′, data processing step 200′, biocarbon treatment step or steps 500′, and output step 600′. Process 10′ also employs first database 203′ and second database 204′, similarly to first database 203 and second database 204, and which store biocarbon material characteristics and concrete performance data, respectively. FIG. 18b shows an example of parameters that are input in step 100′. FIG. 18c shows an example of data that is stored in first database 203′ and/or second database 204′. FIG. 18d shows inputs that can be added to first database 203′ that relate to biocarbon material characteristics.

The present disclosure contemplates that multiple aspects and/or features described herein can be combined, and not necessarily in the order they are described or shown. Further, several method steps or aspects are optional and do not need to be performed. For example, one non-limiting aspect of the present disclosure would include step 100, acquiring an original concrete mixture, sending the original concrete mixture to DOT 202 in step 200, and outputting a revised concrete mixture in step 600, without any of the processing in step 500. Another non-limiting aspect of the disclosure includes acquiring the original concrete mixture, step 100, sending the mixture to DOT 202 in step 200, processing a biocarbon concrete replacement material in step 500, and outputting both the concrete ingredient replacer and revised concrete mixture in step 100. Step 500 could include all of sub-steps 502.1 through 502.5, or only one or more of those sub-steps. For example, pre-wetting step 502.1 can be excluded, as can sub-step 502.2, to adjusting the particle sizes of the biocarbon.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

### CLAUSES

A method of modifying an original concrete mixture, comprising the steps of:
obtaining the original concrete mixture, desired properties of a revised concrete mixture, and desired performance parameters of the revised concrete mixture;
analyzing a first database and a second database to determine a replacement biocarbon as at least one concrete ingredient replacer in the original concrete mixture, wherein the first database comprises physical and chemical properties of a plurality of biocarbons, and the second database comprises performance data for concrete compositions comprising a second plurality of biocarbons; and
determining the revised concrete mixture.

The method of the first clause, further comprising the steps of:
outputting a set of parameters relating to the replacement biocarbon, wherein the set of parameters comprises material properties of the replacement biocarbon; and
outputting the revised concrete mixture, wherein the revised concrete mixture comprises the replacement biocarbon.

The method of any preceding clause, wherein the analyzing step comprises determining the replacement biocarbon by predicting the performance of the revised concrete mixture, based on the information in the first and second databases.

The method of any preceding clause, wherein the predicted performance of the revised concrete mixture matches or exceeds the performance of the original concrete mixture.

The method of any preceding clause, wherein the analyzing step further comprises using machine learning and/or artificial intelligence to perform the predicting step.

The method of any preceding clause, wherein the original concrete mixture comprises a cement and an aggregate, and the replacement biocarbon at least partially replaces at least one of the cement and the aggregate.

The method of any preceding clause, wherein the outputting the set of parameters step comprises sending the material properties of the replacement biocarbon to a manufacturing facility, and wherein the manufacturing facility makes the replacement biocarbon according to the material properties.

The method of any preceding clause, wherein the replacement biocarbon is a mixture of at least two biocarbons.

The method of any preceding clause, wherein a process for making concrete according to the revised concrete mixture emits less carbon than a process for making concrete according to the original concrete mixture.

The method of any preceding clause, wherein a process for making concrete according to the revised concrete mixture is less expensive than a process for making concrete according to the original concrete mixture.

The method of any preceding clause, wherein the first database comprises one or more of the biocarbon material properties listed above with respect to first database 203.

The method of any preceding clause, wherein the second database comprises one or more of the concrete performance indicators listed above with respect to second database 204.

The method of any preceding clause, wherein the outputted parameters include at least one selected from the group consisting of: cement type and or suitability, additives type and dose and or suitability, water/cement ratio and or water correction factor, quantity & blend of biocarbon materials, particle sizes of biocarbon materials, cement percentage replacement, aggregate(s) percentage replacement, CO2 footprint of the revised concrete mixture, concrete performance prediction, and key biocarbon performance indicators, e.g. ash content, specific surface area, carbon content, and silica content.

The method of any preceding clause, wherein the analyzing step comprises determining the mechanical strength performance indicator of a particular biocarbon, by considering at least one of the following factors: feedstock material, average feedstock processing temperature, carbon content, ash content, oxide content, surface morphology and surface charge, moisture content (%) of the biocarbon (partial or water full saturation), pH (at integration and variability over time), and water holding capacity.

The method of any preceding clause, wherein the analyzing step comprises determining the rheological performance indicator of a particular biocarbon, by considering at least one of the following factors: moisture content (%) of the biocarbon (partial or water full saturation), water holding capacity, particle size distribution, particle size and shape, specific surface area, ash content, carbon content, pH (at integration and fluctuations over time), and zeta potential.

The method of any preceding clause, wherein the analyzing step comprises determining the long-term stability performance indicator(s) of a particular biocarbon(s), by considering at least one of the following factors: feedstock material, average feedstock processing temperature, carbon content, ash content, ash composition, oxide content, oxygen/carbon ratio, hydrogen/carbon ratio, surface morphology and surface charge, zeta potential, and pH (at integration and fluctuations over time).

The method of any preceding clause, wherein the analyzing step comprises determining the thermal conductivity performance indicator(s) of a particular biocarbon(s), by considering at least one of the following factors: specific density, specific surface area, carbon content, and ash content.

The method of any preceding clause, wherein the analyzing step comprises determining the CO2 performance indicator(s) of a particular biocarbon(s), by considering at least one of the following factors: carbon content, silica content, and ash content.

A method of producing a concrete mixture according to a specification, wherein the specification is determined according to the revised concrete mixture of the first clause.

A method of producing a concrete mixture according to a specification, wherein the specification is determined according to the output parameters and revised concrete mixture of the second clause.

A concrete mixture produced according to the method of the first clause.

A concrete mixture produced according to the method of the second clause.

A digital optimization tool, comprising:
a first database;
a second database; and
a processor,
wherein the first database comprises data relating to material characteristics of a first plurality of biocarbons,
wherein the second database comprises data relating to performance characteristics of concrete compositions comprising a second plurality of biocarbons,
wherein the processor receives input relating to an original concrete composition, and analyzes the first and second databases to determine a replacement biocarbon for at least one ingredient in the original concrete composition, and outputs a revised concrete composition, wherein the revised concrete composition has improved carbon emission and/or sequestration as compared to the original concrete composition.

The digital optimization tool, wherein the processor uses machine learning and/or artificial intelligence to analyze the first and second databases and determine the replacement carbon.

1-28. (canceled)

29. A method of producing a concrete mixture comprising at least one biocarbon, the method comprising determining a set of parameters for processing the at least one biocarbon and/or the concrete mixture based on a biocarbon water correction factor (BWCF) of the at least one biocarbon, which is defined as a percentage of a water absorption of the at least one biocarbon, wherein the biocarbon water correction factor is preferably in a range of 10 to 100% of the water absorption of the at least one biocarbon, more preferred 40 to 70% of the water absorption when the concrete mixture is a flowable concrete mixture or 20 to 50% of the water absorption, when the concrete mixture is an earth moist concrete mixture.

30. The method according to claim 29, comprising the step of determining a water absorption of the at least one biocarbon before determining the set of parameters, wherein the water absorption is preferably determined by measuring or calculating or estimating taking into account a water holding capacity, a water content, a median particle size and/or particle density of the at least one biocarbon.

31. The method according to claim 29, wherein the set of parameters comprises at least one selected from the group consisting of:
selection of at least one biocarbon from a group of possible biocarbons for the concrete mixture;
required median particle size,
required water content of the at least one biocarbon before adding to the concrete mixture
process parameters for an adjustment of the median particle size,
process parameters for a pre-wetting of the at least one biocarbon,
dose of the selected at least one biocarbon,
required total amount of added water,
required amount of added water during mixing the concrete mixture,
required amount and type of added cement,
required amount and type of at least one additive,
required amount and type of at least one plasticizer, preferably of at least one superplasticizer,
aggregate replacement ratio,
a corrected water-cement ratio,
order of addition.

32. The method according to claim 31, wherein the corrected water-cement ratio is determined as
$${( \frac{w}{c} )}_{corr} = {( \frac{w}{c} )}_{t} \cdot ( 1 + \frac{BWCF \cdot {m}_{biocarbon}}{{w}_{t}} ) ,$$
wherein BWCF is the biocarbon water correction factor,
$${( \frac{w}{c} )}_{t}$$
is a targeted water-cement ratio for a reference concrete mixture without biocarbon, m_{biocarbon} is the mass of biocarbon in the concrete mixture and wₜ is a targeted water amount for the reference concrete mixture without biocarbon
and/or
wherein the corrected water-cement ratio is determined as
$${( \frac{w}{c} )}_{corr} = {( \frac{w}{c} )}_{t} \cdot ( 1 + \frac{BWCF \cdot {m}_{biocarbon}}{{{c}_{t} ( \frac{w}{c} )}_{t}} ) ,$$
wherein BWCF is the biocarbon water correction factor,
$${( \frac{w}{c} )}_{t}$$
iS a targeted water-cement ratio for a reference concrete mixture without biocarbon, m_{biocarbon} is the mass of biocarbon in the concrete mixture and cₜ is a targeted cement amount for the reference concrete mixture without biocarbon
and/or
wherein the required total amount of water is determined as
wₜₒₜₐₗ=wₜ+BWCF·m_{biocarbon} wherein BWCF is the biocarbon water correction factor, wₜ is a targeted water amount according to the targeted water-cement ratio for the reference concrete mixture without biocarbon
$${( \frac{w}{c} )}_{t}$$
and m_{biocarbon} IS the mass of biocarbon in the concrete mixture
and/or
wherein the required amount of cement is determined as
$$c = \frac{( {w}_{t} + BWCF \cdot {m}_{biocarbon} )}{{( \frac{w}{c} )}_{corr}} ,$$
wherein BWCF is the biocarbon water correction factor,
$${( \frac{w}{c} )}_{corr}$$
is the corrected water-cement ratio, m_{biocarbon} is the mass of biocarbon in the concrete mixture and wₜ is a targeted water amount for the reference concrete mixture without biocarbon
and/or
wherein the required total amount of water is determined as a sum of a water content added during pre-wetting the biocarbon and an added water amount during mixing the concrete mixture
and/or
wherein the order of addition comprises blending fine and coarse aggregates, adding 100% of the selected at least one biocarbon to the fine and coarse aggregates, adding at least the amount of water according to the biocarbon water correction factor to the mix of at least one biocarbon and aggregates, adding cement, adding water up to the total amount of water, optionally adding at least one plasticizer, preferably at least one superplasticizer.

33. The method according to claim 29, wherein determining the set of parameters comprises using at least one further material property of the at least one biocarbon, the at least one further material property comprise at least one of
carbon content,
ash content,
oxide content,
random reflectance,
surface morphology and surface charge,
water content (%) (partial or water full saturation),
particle size distribution,
median particle size,
particle size and shape and
specific surface area and/or,
wherein determining the set of parameters additionally comprises using at least one performance parameter of at least one known concrete mixture, wherein the at least one performance parameter comprises
mechanical strength performance comprising 24 h, 7 and 28 day compressive and/or flexural strength and/or
concrete rheological performance comprising workability and/or flowability and/or
slump retention and/or
flow diameter retention and/or
thermal conductivity and/or
fresh density and/or
dry density and/or
setting time and/or
capillary porosity and/or
permeability and/or
long-term stability and/or
carbonation and/or
freeze thaw resistance and/or
air content and/or
fire/heat resistance and/or
shrinkage.

34. The method according to claim 29, wherein in determining the set of parameters it is additionally taken into account
that for a flowable concrete a 28 d strength is optimized with a water content of the biocarbon before adding to the concrete mixture of 10 to 100% of the water absorption, wherein the water content is preferably in the range 40 to 70% of the water absorption of the biocarbon, wherein the 28 d strength is preferably at least in strength class C25/30 and or C30/37, for an earth moist concrete a 28 d strength is optimized with a water content of the biocarbon before adding to the concrete mixture of 10 to 100% of the water absorption, wherein the water content is preferably in the range20-50% of the water absorption of the biocarbon, wherein the 28 d strength is preferably at least 15 MPa and wherein for a lightweight earth moist concrete the 28 d strength is preferably at least 1.5 MPa,
and/or
that a 28 d strength is optimized with a median particle size range of 0.010 mm to 0.5 mm, for a biocarbon dose up to 180 kg/m3,
wherein for a flowable concrete the 28 d strength is preferably optimized with a median particle size range of 0.001 to 0.030 mm and/or with a median particle size of 0.010 mm, in particular containing 99% of particles finer than 0.100 mm, 95% of particles finer than 0.063 mm, 70% of particles finer than 0.025 mm, wherein the 28 d strength for a flowable concrete is preferably at least 30 MPa, and/or
wherein for an earth moist concrete a 28 d strength is preferably optimized with a median particle size range of 0.001 to 0.080 mm and/or with a median particle size of 0.046 mm, in particular containing 99% of particles finer than 2.000 mm, 97% of particles finer than 1.000 mm, 87% of particles finer than 0.500 mm, 72% of particles finer than 0.200 mm, 64% of particles finer than 0.100 mm, 58% of particles finer than 0.063 mm, 32% of particles finer than 0.025 mm, wherein the 28 d strength for an earth moist concrete is preferably at least 15 MPa, and/or
wherein for a lightweight earth moist concrete a 28 d strength is preferably optimized with a median particle size range of 0.100 to 0.500 mm and/or a median particle size of 0.380 mm, in particular containing 99% of particles finer than 4.000 mm, 86% of particles finer than 2.000 mm, 76% of particles finer than 1.000 mm, 58% of particles finer than 0.500 mm, 32% of particles finer than 0.200 mm, 13% of particles finer than 0.100 mm, wherein the 28 d strength is for a lightweight earth moist concrete preferably at least 1.5 MPa
and/or
that a 28 d strength is optimized if at least one additive, comprising preferably a plasticizer, more preferred a superplasticizer, is present in the concrete mixture according to an additive w/c optimization parameter
$${X}_{ADwc} = \frac{{( \frac{w}{c} )}_{corr} \cdot {c}_{AD}}{{d}_{biocarbon}} * 100 , wherein {( \frac{w}{c} )}_{corr}$$
is the corrected water-cement ratio, c_{AD} is a dose of additive in weight-% of a cement content, d_{biocarbon} is the dosage of biocarbon in kg/m3 and X_{ADwc} is in the range of 2.50 to 0.30, preferably between 2.0 and 0.4, more preferably between 1.44 and 0.45
and/or
that a 28 d strength is optimized for an earth moist concrete if water, cement and the at least one biocarbon are present in the concrete mixture according to a biocarbon w/c optimization parameter
$${Y}_{Bwc} = \frac{{( \frac{w}{c} )}_{corr}}{{d}_{biocarbon}} * 100 , wherein {( \frac{w}{c} )}_{corr}$$
is the corrected water-cement ratio and d_{biocarbon} is the dosage of biocarbon in kg/m3 and Y_{Bwc} is in the range of 0.45 and 1.6, preferably between 0.6 and 1.2, more preferably between 0.7 and 1.0
and/or
that for a flowable concrete a 28 d strength is improved using a corrected water-cement ratio between 0.40 and 0.70, preferably 0.45 to 0.68 and for an earth moist concrete a 28d strength is improved using a corrected water-cement ratio between 0.36 and 0.80, preferably 0.40 to 0.65.

35. The method according to claim 29, wherein determining the set of parameters additionally comprises using that with a water content of the biocarbon before adding to the concrete mixture of 25-75% a slump retention and/or flow diameter retention of up to 45 min is maintained
and/or
that a higher water holding capacity or water absorption of a biocarbon leads to a reduction in the flow diameter of concrete,
and/or
that the finer a median particle size the higher is the flow diameter of mortar or concrete, wherein preferably the flow diameter is in a range of 300 to 600 mm, more preferred 350 to 550 mm, for a median particle size of range 0.001 to 0.350 mm, wherein preferably the median particle size is 0.010 mm, 0.046 mm or 0.090 mm,
and/or
that for a targeted slump retention and/or flow diameter retention of 0 to 30 min a polycarboxylate PCE based superplasticizer is favorable, preferably in a dose of 0.2 to 3.0%, more preferred 0.4 to 1.2%, and for targeted slump retention and/or flow diameter retention of 30 to 60 min a lignosulfonate based superplasticizer is favorable, preferably in a dose of 0.2 to 1.5%, more preferred 0.3 to 1.1%.

36. The method according to claim 29, further comprising at least one step of processing the at least one biocarbon and/or the concrete mixture using the set of parameters, in particular at least one of the following processing steps:
selecting at least one biocarbon of the group of possible biocarbons for adding into the concrete mixture and/or
treating the at least one biocarbon preferably via at least pre-wetting and/or adjustment of the median particle size and/or
dosing the at least one biocarbon and/or
adding water and/or
adding cement and/or
adding at least one additive, in particular at least one plasticizer, in particular at least one superplasticizer.

37. The method according to claim 36, wherein water and/or cement is added into the concrete mixture based on the biocarbon water correction factor, preferably with the biocarbon water correction factor being in the range of 10 to 100% of the water absorption, more preferred 40-70% of the water absorption of the at least one biocarbon for flowable concrete mixture or 20-50% of the water absorption for an earth moist concrete mixture
and/or
wherein water and/or cement is added into the concrete mixture according to the corrected water-cement ratio being for a flowable concrete between 0.40 and 0.70, preferably 0.45 to 0.68 and for an earth moist concrete being between 0.36 and 0.80, preferably 0.40 to 0.65
and/or
wherein for an earth moist concrete water, cement and the at least one biocarbon are added in the concrete mixture according to the biocarbon w/c optimization parameter
$${Y}_{Bwc} = \frac{{( \frac{w}{c} )}_{corr}}{{d}_{biocarbon}} * 100 , wherein {( \frac{w}{c} )}_{corr}$$
is the corrected water-cement ratio and d_{biocarbon} is the dosage of biocarbon in kg/m3 and Y_{Bwc} is in the range of 0.45 and 1.6, preferably between 0.6 and 1.2, more preferably between 0.7 and 1.0.

38. The method according to claim 36, wherein water and/or cement is added into the concrete mixture according to the corrected water-cement ratio
$${( \frac{w}{c} )}_{corr} = {( \frac{w}{c} )}_{t} \cdot ( 1 + \frac{BWCF \cdot {m}_{biocarbon}}{{w}_{t}} ) ,$$
wherein BWCF is the biocarbon water correction factor,
$${( \frac{w}{c} )}_{t}$$
is a targeted water-cement ratio for a reference concrete mixture without biocarbon, m_{biocarbon} is the mass of biocarbon in the concrete mixture and wₜ is a targeted water amount for the reference concrete mixture without biocarbon
and/or
according to
$${( \frac{w}{c} )}_{corr} = {( \frac{w}{c} )}_{t} \cdot ( 1 + \frac{BWCF \cdot {m}_{biocarbon}}{{{c}_{t} ( \frac{w}{c} )}_{t}} ) ,$$
wherein BWCF is the biocarbon water correction factor,
$${( \frac{w}{c} )}_{t}$$
is a targeted water-cement ratio for a reference concrete mixture without biocarbon, m_{biocarbon} is the mass of biocarbon in the concrete mixture and cris a targeted cement amount for the reference concrete mixture without biocarbon
and/or
according to wₜₒₜₐₗ=wₜ+BWCF·m_{biocarbon}, wherein BWCF is the biocarbon water correction factor, wₜ is a targeted water amount according to the targeted water-cement ratio for the reference concrete mixture without biocarbon
$${( \frac{w}{c} )}_{t}$$
and m_{biocarbon} IS the mass of biocarbon in the concrete mixture
and/or
according to
$$c = \frac{( {w}_{t} + BWCF \cdot {m}_{biocarbon} )}{{( \frac{w}{c} )}_{corr}} ,$$
wherein BWCF is the biocarbon water correction factor,
$${( \frac{w}{c} )}_{corr}$$
is the corrected water-cement ratio, m_{biocarbon} is the mass of biocarbon in the concrete mixture and wₜ is a targeted water amount for the reference concrete mixture without biocarbon
and/or
wherein the required total amount of water is a sum of a water content added during pre-wetting the biocarbon and an added water amount during mixing the concrete mixture.

39. The method according to claim 36, wherein the at least one biocarbon is pre-wetted to a water content of 10-100% of the water absorption of the at least one biocarbon, preferably pre-wetted to a water content in the range of 10-75, more preferred in the range of 10-50%, even more preferred in the range of 10 to 25% of the water absorption of the at least one biocarbon for a flowable concrete or to a water content of 10 to 80, more preferred in the range of 10 to 60%, even more preferred in the range of 40 to 60% of the water absorption of the at least one biocarbon for an earth moist concrete.

40. The method according to claim 36, wherein the median particle size range of the at least one biocarbon is adjusted to a range of 0.001 mm to 0.5 mm,
preferably for a flowable concrete mixture to a median particle size range of 0.001 to 0.030 mm and/or to a median particle size of 0.010 mm, in particular containing 99% of particles finer than 0.100 mm, 95% of particles finer than 0.063 mm, 70% of particles finer than 0.025 mm, or
for an earth moist concrete mixture to a median particle size range of 0.001 to 0.080 mm and/or to a median particle size of 0.046 mm, in particular containing 99% of particles finer than 2.000 mm, 97% of particles finer than 1.000 mm, 87% of particles finer than 0.500 mm, 72% of particles finer than 0.200 mm, 64% of particles finer than 0.100 mm, 58% of particles finer than 0.063 mm, 32% of particles finer than 0.025 mm, or
for a lightweight earth moist concrete mixture to a median particle size range of 0.100 to 0.500 mm and/or to a median particle size of 0.380 mm, in particular containing 99% of particles finer than 4.000 mm, 86% of particles finer than 2.000 mm, 76% of particles finer than 1.000 mm, 58% of particles finer than 0.500 mm, 32% of particles finer than 0.200 mm, 13% of particles finer than 0.100 mm.

41. The method according to claim 36, wherein the order of addition is blending fine and coarse aggregates, adding 100% of the at least one biocarbon to the fine and coarse aggregates, adding at least the amount of water according to the biocarbon water correction factor to the mix of at least one biocarbon and aggregates, adding cement, adding water up to the total amount of water, optionally adding at least one plasticizer, preferably at least one superplasticizer, and/or,
wherein at least one additive, comprising preferably a plasticizer, more preferred a superplasticizer, is added into the concrete mixture according to the additive w/c optimization parameter
$${X}_{ADwc} = \frac{{( \frac{w}{c} )}_{corr} \cdot {c}_{AD}}{{d}_{biocarbon}} * 100 , wherein {( \frac{w}{c} )}_{corr}$$
is the corrected water-cement ratio, c_{AD} is a dose of additive in weight-% of a cement content, d_{biocarbon} is the dosage of biocarbon in kg/m3, X_{ADwc} being in the range of 2.50 to 0.30, preferably between 2.0 and 0.4, more preferably between 1.44 and 0.45
and/or
wherein a polycarboxylate (PCE) based superplasticizer, preferably in a dose of 0.2 to 3.0%, more preferred 0.4 to 1.2%, is added to the concrete mixture and/or wherein a lignosulfonate based superplasticizer preferably in a range of 0.2 to 1.5%, more preferred 0.3 to 1.1%, is added to the concrete mixture.

42. A concrete mixture or an intermediate product, in particular a customized biocarbon, produced or processed according to the method of claim 29.

43. A customized biocarbon, in particular processed according to the method of claim 29, for use in flowable concrete having
a water content of 10-100%, preferably of 40-70% of the water absorption of the biocarbon and
a median particle size range of 0.001 mm to 0.5 mm, preferably of 0.001 to 0.030 mm, preferably having a median particle size of 0.010 mm, in particular containing 99% of particles finer than 0.100 mm, 95% of particles finer than 0.063 mm, 70% of particles finer than 0.025 mm or
for use in earth moist concrete having
a water content of 10-100%, preferably of 20-50% of the water absorption of the biocarbon and
a median particle size range of 0.001 mm to 0.5 mm, preferably of 0.001 to 0.080 mm, preferably having a median particle size of 0.046 mm, in particular containing 99% of particles finer than 2.000 mm, 97% of particles finer than 1.000 mm, 87% of particles finer than 0.500 mm, 72% of particles finer than 0.200 mm, 64% of particles finer than 0.100 mm, 58% of particles finer than 0.063 mm, 32% of particles finer than 0.025 mm or for use in lightweight earth moist concrete having
a water content of 10-100%, preferably of 20-50% of the water absorption of the biocarbon and
a median particle size range of 0.001 mm to 0.5 mm, preferably of 0.100 to 0.500 mm, preferably a median particle size of 0.38 mm, in particular containing 99% of particles finer than 4.000 mm, 86% of particles finer than 2.000 mm, 76% of particles finer than 1.000 mm, 58% of particles finer than 0.500 mm, 32% of particles finer than 0.200 mm, 13% of particles finer than 0.100 mm.

44. Use of a biocarbon according to claim 43 in flowable concrete, earth moist concrete or lightweight earth moist concrete.

45. A concrete mixture comprising biocarbon, in particular produced according to the method of claim 29, having a corrected water-cement ratio of
$${( \frac{w}{c} )}_{corr} = \frac{{w}_{t} + BWCF \cdot {m}_{biocarbon}}{c} ,$$
wherein BWCF is the biocarbon water correction factor, m_{biocarbon} IS the mass of biocarbon in the concrete mixture c is the cement amount in the concrete mixture and wₜ is a targeted water amount for a reference concrete mixture without biocarbon and wherein BWCF is in a range of 10-100% of the water absorption of the at least one biocarbon, more preferred of 40 to 70% of the water absorption when the concrete mixture is a flowable concrete mixture or of 20 to 50% of the water absorption when the concrete mixture is an earth moist concrete mixture.

46. The concrete mixture according to claim 45, comprising at least one additive, comprising preferably a plasticizer, more preferred a superplasticizer according to the additive w/c optimization parameter
$${X}_{ADwc} = \frac{{( \frac{w}{c} )}_{corr} \cdot {c}_{AD}}{{d}_{biocarbon}} * 100 , wherein {( \frac{w}{c} )}_{corr}$$
is the corrected water-cement ratio, c_{AD} is a dose of additive in weight-% of a cement content, d_{biocarbon} is the dosage of biocarbon in kg/m3 and X_{ADwc} is in the range of 2.50 to 0.30, preferably between 2.0 and 0.4, more preferably between 1.44 and 0.45
and/or
comprising water, cement and the at least one according biocarbon to the biocarbon w/c optimization parameter
$${Y}_{Bwc} = \frac{{( \frac{w}{c} )}_{corr}}{{d}_{biocarbon}} * 100 , wherein {( \frac{w}{c} )}_{corr}$$
is the corrected water-cement ratio and d_{biocarbon} is the dosage of biocarbon in kg/m3 and Y_{Bwc} is in the range of 0.45 and 1.6, preferably between 0.6 and 1.2, more preferably between 0.7 and 1.0
and/or
wherein the corrected water-cement ratio is for a flowable concrete between 0.40 and 0.70, preferably 0.45 to 0.68 and is for an earth moist concrete between 0.36 and 0.80, preferably 0.40 to 0.65.

47. The concrete mixture according to claim 45, in particular a flowable concrete mixture, with a dosage of biocarbon of 60 kg/m³ to 180 kg/m³ having a 28 d compressive strength at least in strength class C25/30 and or C30/37 and/or a fresh density of at least 2100 kg/m³ or in particular an earth moist concrete mixture, with a dosage of biocarbon of 60 kg/m³ to 180 kg/m³ having a 28 d compressive strength of at least 15 MPa and/or a fresh density of at least 2100 kg/m³.

48. A computer readable medium carrying a computer program comprising program code means for performing the method according to claim 29, when said program product is run on a computer.
